(19) United States
(12) Patent Application Publication
(10) Pub. No.: US 2026/0325001 A1
(43) Pub. Date: Oct. 22, 2026

(54) LIGHTWEIGHT VIDEO MRI ASSEMBLY

(71) Applicants: David MICHAELI, Ashkelon (IL); Menashe MICHAELI, Ashkelon (IL); Sviatoslav TARASENKO, Istanbul (TR)

(72) Inventors: David MICHAELI, Ashkelon (IL); Menashe MICHAELI, Ashkelon (IL); Sviatoslav TARASENKO, Istanbul (TR)

(21) Appl. No.: 19/696,364

(22) Filed: Jun. 3, 2026

Related U.S. Application Data
(63) Continuation-in-part of application No. PCT/IB2025/062065, filed on Nov. 25, 2025.
(60) Provisional application No. 63/726,297, filed on Nov. 28, 2024.
(60) Provisional application No. 63/726,302, filed on Nov. 28, 2024.

(51) Int. Cl. A61B 5/055; A61B 5/00; G01R 33/34

(52) U.S. Cl. CPC A61B 5/055; A61B 5/0042; A61B 5/0044; G01R 33/34023; G01R 33/3403; G01R 33/34084

(57) ABSTRACT

An assembly for an MRI video imaging system has electromagnets including a coil assembly that may have multiple segments independently connected to a power supply. The independent power supply, controlled by the controller, and connected to each of the electromagnets includes piezoelectric material and generates pulses of electric current at a specified repetition rate and for a specified duration. Detectors detect an NMR signal as a result of the tissue of the subject being under an influence of the electromagnetic field, and transmit the NMR signal to a computer system to generate a video image. The imaging session does not exceed (i) 90 seconds for a video MRI of the whole body or (ii) 30 seconds for a video MRI of an organ or region. Still images corresponding to multiple (or most or all) MRI protocols of a region or organ may be extracted from the video in some embodiments.

### FIELD OF THE INVENTION

The embodiments described herein generally relate to an assembly for a lightweight MRI imaging system or machine, and more particularly, in some embodiments, to an assembly for MRI imaging systems that utilizes piezoelectric material and the emission of brief pulses of electricity in a power supply for its electromagnets, for example to achieve a video output.

### BACKGROUND

There are two types of stationary MRI imaging systems-those that use permanent magnets and those that use superconductive magnets. As of 2023 about 70% of MRI imaging systems installed worldwide use superconducting magnets. These machines weigh 4,500 Kgs (1.5 T) or 7,500 Kgs (3.0 T). Other MRI imaging machines use permanent magnets, which weigh up to 16,000 Kgs. Both types of MRI imaging systems expend a lot of energy and generate a lot of heat. They therefore require built-in cooling systems involving many heavy condensers.

Besides the weight, other drawbacks of MRI imaging systems include their much higher cost, and patient discomfort with the procedure. The electromagnets of an MRI machine are also very powerful and necessitate shielding the whole room in which the MRI scanning occurs.

MRI imaging systems are also not as accessible as they could be. An MRI scan can only be done in a hospital and typically requires scheduling well in advance.

There is a portable MRI imaging system for the head produced by Hyperfine®, Inc. called the Swoop® system. The company states that it weighs about 635 Kgs (1,400 pounds). It is not designed to be used outside of a hospital, although it claims to be wheelable to the patient within the hospital. It utilizes a permanent magnet having a magnetic field strength of 64 mT (milli Tesla). Its claimed operating temperature range is 15 to 30 degrees Celsius. However, the quality of its brain imaging is believed by Applicant to not be of high quality because the use of an outside magnetic field strength of 64 milli Tesla means that the flip angle of the protons (in the tissue of the patient) exhibiting precession is low. As a result, the MRI signals emitted from the brain tissue using the Hyperfine® system are of low intensity, and have a low signal to noise ratio. The resulting brain imaging has low contrast and spatial resolution. The low contrast and spatial resolution of the brain images is further exacerbated by the absence of a cooling system, which elevates the noise signals and lowers the level of beneficial MRI signals. It is noted that the assembly described below is not limited to any particular field strength, and the foregoing is provided as illustrative context only.

### SUMMARY OF THE EMBODIMENTS

One embodiment is an assembly for an MRI video imaging system, the assembly comprising:
a subject-receiving region having at least one surface;
electromagnets formed as a coil assembly and configured to generate an electromagnetic field, the coil assembly configured to emit a radio frequency (RF) signal to the subject;
a power supply connected to the electromagnets and configured to generate pulses of electric current, the power supply comprising piezoelectric material and an actuator, and controlled by a controller so as to generate the pulses of electric current at a repetition rate and for a duration specified by the controller;
detectors configured to detect a nuclear magnetic resonance (NMR) signal from tissue of the subject, and to transmit the NMR signal for use by a computer system in generating a video image that implements multiple MRI protocols,
wherein each coil assembly comprises at least two inter-nested coils, each of the at least two inter-nested coils configured to carry pulses of electric current at the same or a different electromagnetic frequency and including an outer coil and at least a first inner coil, such that the outer coil of one particular coil assembly at least partially shields the electromagnetic fields generated by at least the first inner coil of the one particular coil assembly from background electromagnetic fields of coils of other adjacent coil assemblies,
each coil assembly has at least two segments, each of the segments connected independently to the power supply.

In some embodiments, each of the coil assemblies comprise three or four inter-nested coils.

In some embodiments, the video image includes at least one full MRI protocol of a whole body of the subject and a duration of an imaging session during which the NMR signals used to generate the video image are acquired does not exceed 90 seconds.

In some embodiments, the video image covers at least one full MRI protocol of an organ or a region of the subject and a duration of an imaging session during which the NMR signals used to generate the video image are acquired does not exceed 30 seconds.

In some embodiments, each coil assembly has from two to twelve segments.

In some embodiments, the power supply is configured to generate the pulses at a particular repetition rate from 1000 to 10000 pulses per second.

In some embodiments, the power supply is configured to produce a current of 10 to 399 amperes.

In some embodiments, the power supply is configured to produce a voltage of from 10 volts to 10 kilovolts.

In some embodiments, the power supply is configured to generate 1-3000 kilowatts.

In some embodiments, a frequency of pulses of electric current transmitted to each coil assembly of the electromagnets is from 100 KHz to 10 MHz.

In some embodiments, the duration of each of the generated pulses is ten milliseconds or less.

In some embodiments, the duration of each of the generated pulses is 3.3 milliseconds to 333 milliseconds.

In some embodiments, a repetition rate of the pulses of the electric current transmitted to each coil assembly of the electromagnets is independently controlled by the controller.

In some embodiments, each of the coils of the electromagnets is shaped substantially frustoconical or shaped substantially as a triangular pyramid whose vertex has been truncated.

In some embodiments, the assembly is a portable assembly and a total weight of the portable assembly is less than 100 kilograms.

In some embodiments, the assembly is a portable assembly and a total weight of the portable assembly is less than 30 kilograms.

In some embodiments, the assembly comprises one or more of the following modules and a total weight of one of the following modules is less than 10 kilograms: (i) a head module, (ii) an abdominal module, (iii) a thorax module and (iv) a legs module.

In some embodiments, the assembly further comprises a cooling fluid flowing through the coil or coil assembly of the electromagnets and further comprising metal nanoparticles in the fluid configured to increase a signal to noise ratio for the transmitted signal.

In some embodiments, the power supply comprises a separate element of piezoelectric material for each of the electromagnets.

Another embodiment is a method of generating a video MRI that includes multiple MRI protocols, comprising: imaging a subject using an MRI assembly that includes a coil assembly of segmented electromagnets of inter-nested coils, a power supply, including a piezoelectric material and an actuator, independently connected to each segment of the segmented electromagnets and configured to generate pulses of electric current at a repetition rate of five-hundred or more pulses per second, a controller controlling the repetition rate and duration of the pulses; and outputting, by a computer system, a video MRI comprising multiple MRI protocols based on NMR signals detected during the imaging.

In some embodiments, each of the coil assemblies comprise at least two inter-nested coils, each of the at least two inter-nested coils of each of the coil assemblies includes an outer coil and at least a first inner coil and is configured to carry pulses of electric current at a same or a different electromagnetic frequency, such that the outer coil of one particular coil assembly at least partially shields the electromagnetic fields generated by at least the first inner coil of the one particular coil assembly from background electromagnetic fields of coils of other adjacent coil assemblies.

In some embodiments, the method further comprises extracting from the video MRI a particular MRI protocol.

In some embodiments, the video MRI comprises at least seven different MRI protocols that are neurological or brain protocols.

In some embodiments, the video MRI encompasses at least four different MRI protocols that are musculoskeletal protocols.

In some embodiments, the video MRI encompasses at least four different MRI protocols that are abdomen and pelvis protocols.

In some embodiments, the video MRI encompasses at least five different MRI protocols that are head, neck or vascular protocols.

In some embodiments, the video MRI encompasses at least five different MRI protocols that are cardiac protocols.

In some embodiments, the video MRI encompasses at least two different MRI protocols that are breast protocols.

In some embodiments, a duration of an imaging session during which the NMR signals used to generate the video image are acquired does not exceed 90 seconds and the video MRI is of a whole body of the subject.

In some embodiments, a duration of an imaging session during which the NMR signals used to generate the video image are acquired does not exceed 30 seconds and the video MRI is of an organ or a region of the subject.

### BRIEF DESCRIPTION OF THE DRAWINGS

Various embodiments are herein described, by way of example only, with reference to the accompanying drawings, wherein;

FIG. 1A is a schematic view of a head module of scanner bore of an assembly for a portable MRI imaging system showing a patient inside part of a scanner bore, in accordance with one embodiment;

FIG. 1B is a schematic view of several module of the scanner bore of an assembly for a portable MRI imaging system showing a patient inside part of a scanner bore, in accordance with one embodiment;

FIG. 1C is a schematic of a portable assembly for an MRI imaging system containing multiple modules that are configured to function independently and/or simultaneously, in accordance with one embodiment;

FIG. 1D is a schematic of a portable assembly showing an adjustable scanner bore for the head module, in accordance with one embodiment;

FIG. 1E is a top view of the bottom layer of a cover with embedded detectors for use in a portable assembly for a head module, in accordance with one embodiment;

FIG. 1F is a schematic view from the side and front of a subject covered by a cover with embedded detectors for use in a portable assembly for a head module, in accordance with one embodiment;

FIG. 1G is a schematic view from the side of a subject covered by a cover with embedded detectors for use in a portable assembly for multiple modules, in accordance with one embodiment;

FIG. 1H1 is a schematic view showing a configuration of electromagnetic coils that are arranged in a curved configuration, including multiple pairs, situated so as to generate an initial three-dimensional image, in accordance with one embodiment;

FIG. 1H2 is a schematic view showing a configuration of electromagnetic coils that includes multiple arrays situated so as to generate an initial three-dimensional image in conjunction with a movable support structure, in accordance with one embodiment;

FIG. 2A is a schematic horizontal sectional view of the thoracic module of the scanner bore and depicting a series of electromagnets in the form of truncated pyramids or frustoconically shaped electromagnets, in accordance with one embodiment;

FIG. 2B is a schematic top view of a series of electromagnets used in the thoracic module of the assembly of FIG. 2A, in accordance with one embodiment;

FIG. 2C1 is a schematic illustration of a coil assembly for an electromagnet including an outer coil and three inner coils, in accordance with one embodiment;

FIG. 2C2 is a schematic illustration of a coil assembly for an electromagnet including an outer coil and two inner coils, in accordance with one embodiment;

FIG. 2D is a schematic illustration of a coil assembly having six segments in each of the coils, in accordance with one embodiment;

FIG. 3A is a schematic horizontal sectional view of the thoracic module of the scanner bore and depicting a series of electromagnets in an alternative configuration of the truncated pyramids, in accordance with one embodiment;

FIG. 3B is a schematic top view of the alternative configuration of the series of electromagnets used in the thoracic module of the assembly of FIG. 3A, in accordance with one embodiment;

FIG. 4 is a schematic view of the coils of an electromagnet used in the assembly for a portable MRI imaging system, in accordance with one embodiment;

FIG. 5 is a schematic view of a truncated pyramidal shape of a core of an electromagnet around which the coils are wrapped to form the truncated pyramidal electromagnet used in an assembly for an MRI imaging system, in accordance with one embodiment;

FIG. 6 is a schematic illustration showing a cooling liquid supply system with galvanic isolation from the coils and a pyro-piezoelectric power supply, for a portable MRI imaging system, in accordance with one embodiment;

FIG. 7 is a workflow of an assembly for a portable MRI machine or system, in accordance with one embodiment;

FIG. 8A is a partial longitudinal cross-sectional view of a coil tube of an electromagnet that includes graphene, in accordance with one embodiment;

FIG. 8B is a longitudinal cross-sectional view of a coil of a superconductive ceramic electromagnet that includes yttrium barium copper oxide, in accordance with one embodiment;

FIG. 8C is a longitudinal cross-sectional view of a coil tube of an electromagnet that has a variable cross-section, in accordance with one embodiment;

FIG. 9 is a flow chart showing a method, in accordance with one embodiment;

FIG. 10 is a flow chart showing a method of producing a superconductive electromagnet coil, in accordance with one embodiment;

FIG. 11 is a flow chart showing a method of MRI imaging, in accordance with one embodiment; and

FIG. 12 is a flow chart showing a method of MRI imaging that outputs an MRI video, in accordance with one embodiment.

### DETAILED DESCRIPTION OF THE EMBODIMENTS

The following detailed description is of the best currently contemplated modes of carrying out the invention. The description is not to be taken in a limiting sense, but is made merely for the purpose of illustrating the general principles of the invention, since the scope of the invention is best defined by the appended claims.

Applicant has discovered that it is possible to have a portable MRI imaging system that is lightweight and compact enough to be conveniently placed and used in (i) an ambulance and other land vehicles, aircraft and spacecraft (ii) a military vehicle on the battlefield such as an armored personnel carrier and (iii) in a person's home. In contrast, the prior art stationary and portable MRI systems cannot be used in these environments.

Applicant has also discovered that it is possible and useful to have MRI imaging systems that are tailored to the individual patient's size, in contrast to the prior art.

The superconducting magnet systems and the permanent magnet systems of static MRI imaging systems generate a lot of heat and are very heavy (and expensive). Furthermore, current portable MRIs weigh at least 635 Kgs. Current MRIs-whether stationary or portable-also do not have scanner bore that are adjustable in diameter (or length). Consequently, a small or very young person's scanned body or body part would be too far away from the emitters to generate images of the highest quality.

Applicant has further determined that the water distribution within a person's body varies from person to person and over the course of time. It also varies from one part of the body of a particular person to another part of that person's body. In addition, it varies over time within the same human body part. Applicant believes that it would be useful to have MRI imaging systems that are tailored to the patient's own distribution of water within their body. Since the hydrogen ion of the water molecule is used by MRI imaging systems as an indicia for the presence of body tissue, and since the water distribution varies over time and varies spatially within a person's body and from person to person, the frequency (or resonance frequency) emitted by the MRI system for scanning a subject's body or organ can be tailored to the actual water distribution of that organ or person.

According to certain embodiments, the portable MRI imaging system assembly of the embodiments herein weighs 100 Kgs or less, for example in some embodiments 80 Kgs or 80-100 Kgs or 100 Kgs. In certain embodiments, MRI imaging system assembly can be as low as seven Kgs by exploiting the use of graphene or an alloy of graphene and metal instead of metal.

In contrast to the prior art, the portable assembly for the MRI imaging system does not utilize a permanent magnet. It does not generate constant electricity but rather pulses of electricity for short periods of time such as 3.3 milliseconds up to 333 milliseconds or less than 100 milliseconds (or 20-40 milliseconds). In one non-limiting version, from 3 to 300 pulses per second are utilized. The use of brief pulses of electricity results in far less heat being generated. This in turn results in a much simpler cooling system, and in some embodiments a cooling system without any condensers. This reduces the weight (and volume) even further. It further reduces the length in each dimension and the maximum length in any dimension. The “maximum length in any dimension” means the magnitude of the length of the dimension that has the greatest length from among the height, width and length.

A “break” is an interval during which the electric current is off, i.e. there is no pulse of electric current. The interval is either bounded by two pulses (one before and one after the interval) or, if the pulse is at the beginning or at the end of the period being examined, the interval is bounded on one side by a pulse and on the other side by the beginning or the end of the measurement period, as the case may be.

It is also imperative to check the exact location of a defect in the brain before initiating neuronal stem cell cellular rehabilitation for stroke victims. Precision is critical for achieving the best rehabilitation outcomes.

MRI imaging systems do not currently produce 3-D images of the brain without first reconstructing 2-D slices. Rather, they take images in planes and the resulting 2D images are reconstructed into a 3-D model. Even alternative technologies such as Diffusion MRI (dMRI) and Functional MRI (fMRI) that provide additional information also require pre-processing and reconstruction of the data, which still requires producing intermediate 2-D slices.

In prior art MRI imaging systems, an initial image is in 2-dimensions and the image is then converted to 3-dimensions. This has numerous drawbacks. One is that the conversion software makes a well-known error called a partial voluming artifact. This is a common artifact in MRI imaging that occurs when a voxel within the image contains a mixture of different tissue types or structures. This affects the process of constructing a 3-D image from the 2-D image. This error has implications in being able to precisely localize the defect such as the location of a blood clot caused by a cerebral vascular accident (CVA) (stroke) as a prerequisite to implementing neuronal stem cell cellular rehabilitation. 2-D images may contain noise or artifacts that can worsen the reconstruction. It is impossible to completely eliminate these unwanted effects and they can negatively affect the final quality of the 3-D image.

Another drawback is the information loss from starting with 2-D images since the 2-D images may not contain all the necessary information about the spatial arrangement of brain structures. If the 2-D images do not cover the entire brain volume or have insufficient resolution, this may reduce the accuracy of 3-D reconstruction, which can lead to distortion or loss of important details. Deformation of structures may also occur during reconstruction, especially if there are significant changes in shape or contour when moving from 2-D to 3-D. A still further drawback is that the process of reconstructing a 3-D image based on 2-D slices requires complex processing algorithms and can be labor-intensive, increasing the risk of errors. A still further drawback is that structural variations between individuals may not be accounted for in the reconstruction process, which may result in models that do not reflect the anatomical features of a particular patient. Yet another drawback is that 3-D reconstruction results may require additional validation using other imaging modalities, which adds steps to the analysis process and increases the overall time and resource costs.

Applicant has discovered a way of reducing this artifact in MRI imaging and avoiding the other drawbacks. This helps creating more precise localization needed for the rehabilitation. This improved localization also has implications in other neurological conditions as well, including Alzheimer's and movement disorders.

The portable MRI scanning assembly, in some embodiments, does not include the computer system that creates the image and/or analyzes the image or diagnoses the patient. The portable MRI imaging assembly is instead connected to remote computer systems that provide the images and/or analysis of the data outputted by the assembly. However, in certain other embodiments, the computer system is not remote.

The portable assembly for the MRI imaging system, in some embodiments, is suitable for use by nonphysicians. Certain embodiments that achieve a very low weight such as less than 10 Kgs can be used virtually anywhere.

In certain embodiments, it is possible to maintain the quality of the MRI image comparable to a 3T stationary MRI machine while dramatically reducing the weight so as to make it accessible to battlefield vehicles, ambulances and even homes. Furthermore in some embodiments, the quality is simultaneously maintained while avoiding having to shield the room or vehicle containing the MRI assembly.

In certain embodiments, for example those in which the frequency emitted by the electromagnets is customized in accordance with the water concentration of the subject or the subject's body part or body portion, the quality may be even greater than that of a stationary MRI machine having a 3T magnet.

In some embodiments, the MRI assembly 10 and system is lightweight and achieves extreme mobility and provides more accessibility that prior art MRI imaging systems. For example, assembly 10 may have a weight as low as 7-16 kg. Assembly 10 may fit into a regular backpack. Assembly 10 maybe easily carried even by an untrained person, robot or dog and in some versions may cost orders of magnitude less than a stationary MRI imaging system, less than the Hyperfine® portable system with much better quality. The assembly 10 or system may also take about one minute to deploy and may have its own independent power supply, thereby being independent of, and avoiding the need for, stationary power sources (as a result of the set of piezoelectric power supply elements), a feature that is valuable in hard-to-reach and extreme conditions.

In some embodiments, assembly 10 is portable and/or is usable in portable MRI machines. In some embodiments, assembly 10 is stationary and/or usable in stationary MRI machines. In some embodiments, assembly 10 is disposable.

Applicant has also noted that when a physician in a particular specialty orders an MRI, that specialist often does so in accordance with the protocol corresponding to the suspected diagnosis. The technician is told by the physician who orders the MRI what MRI protocol the physician is interested in. For example if the neurosurgeon suspects a growth the protocol will be for a suspected intracranial growth. If no growth is found, oftentimes the patient five or ten years later complains of a loss of memory and various kinds of dementia are suspected, such as Alzheimer's. When this new symptom appears, a new MRI may well be needed. The previously performed MRI, having been tailored for a suspected intracranial growth, will not be tailored toward and will not satisfy the demands of an Alzheimer's protocol. The Alzheimer's MRI protocol for example requires particular attention being paid to imaging of particular arteries and the areas surrounding them, and it also requires particularly thin imaging slices (for example one millimeter or half a millimeter), something that the intracranial growth MRI protocol may not satisfy.

The above is just one of many examples where this happens since every MRI procedure has its own protocol. Certain embodiments herein yield a video collection of images rather than static images. In certain embodiments of the MRI assembly herein the number of frames per second is a hundred, or in other embodiments is a thousand or in other embodiments is even multiple thousands. Using known software, still images can be extracted from the video. In certain embodiments, the video may contain data sufficient to reconstruct still images corresponding to a plurality of MRI protocols for the imaged region (or whole body of the subject). In some other embodiments, the video may contain data sufficient to reconstruct still images corresponding to most of the MRI protocols for the imaged region (or whole body of the subject). In certain embodiments, the video may contain data sufficient to reconstruct still images corresponding to even all of the MRI protocols for the imaged region (or whole body of the subject)

One of the advantages of achieving a video MRI output is that if a patient who was imaged (with a video MRI output) due to one symptom which without a video MRI would demand a first MRI protocol, later experiences (for example years later) a second symptom that would demand a second MRI protocol, the video MRI output can be used at that later time to extract the still images corresponding with the second protocol. In other words, in certain embodiments, the video MRI output can be prepared based on all MRI protocols for that region of the body.

A yet further advantage of the video MRI in certain embodiments herein is its shorter duration. It is well known that MRI imaging sessions have a drawback in that they are uncomfortable due to claustrophobia and due to their length of time (relative to other imaging sessions such as ultrasound and CT). Certain embodiments disclosed herein can output a video MRI for the whole body of the patient in 60-90 seconds. Certain embodiment of the video MRI disclosed herein can output a video MRI for a particular organ or region of the body (in one example the brain) in approximately 30 seconds or even 15 seconds in certain cases. This means that the length of the imaging session that the patient experiences is 60-90 seconds for whole body imaging in certain embodiments and 15-30 seconds for imaging of regions or organs. While certain prior art MRIs claim to be able to be performed on the patient in one to five minutes, these are highly specific targeted MRI protocols geared for limited medical conditions and are not available for the overwhelming majority of medical conditions.

An “MRI protocol” as used herein refers to protocols for MRI imaging sessions of a human subject or patient that have been accepted by the medical profession as standard for that anatomical system, organ, body region or the whole body of the human subject.

While certain embodiments are configured for portable MRI assemblies or portable use, the assembly 10 is also suitable for stationary MRI applications. “Resonance frequency” refers to a frequency of the signal emitted from the body tissue of the subject (during an MRI imaging process in response to the signal from the MRI imaging assembly) that is the frequency of the highest amplitude. “About” means plus or minus 10%.

The principles and operation of a Lightweight Video MRI Assembly according to the invention may be better understood with reference to the drawings and the accompanying description.

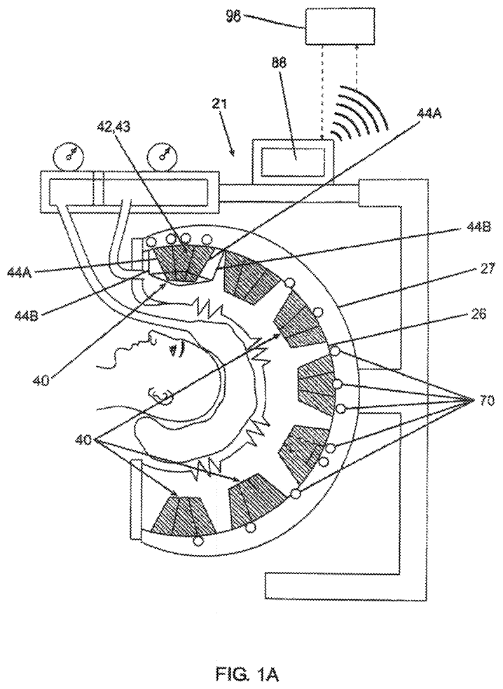

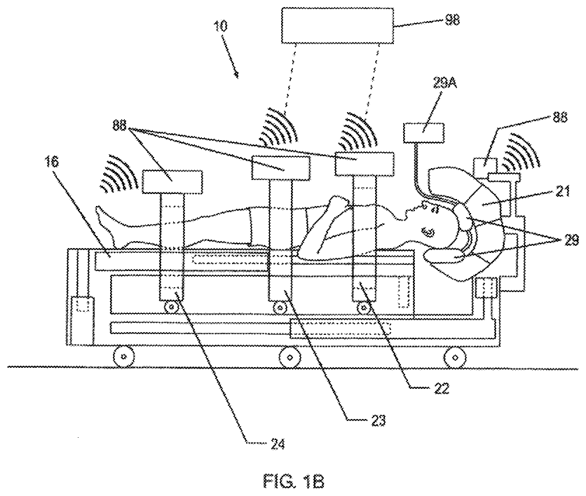

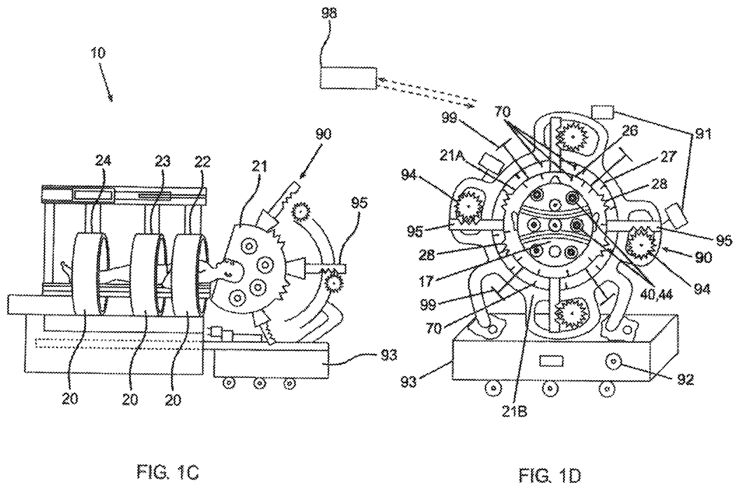

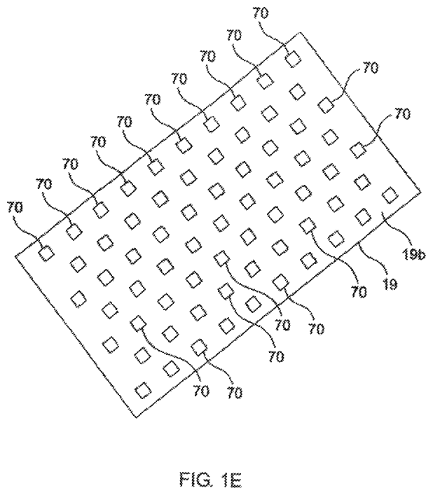

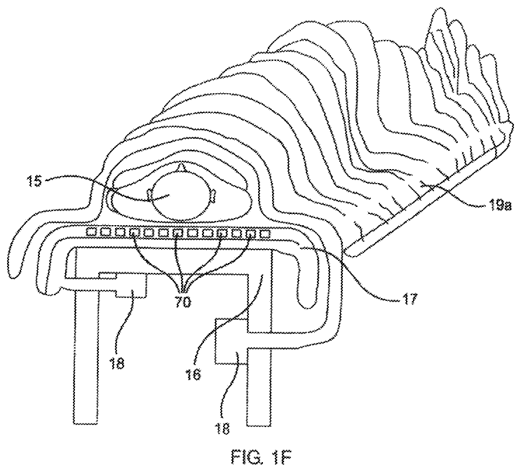

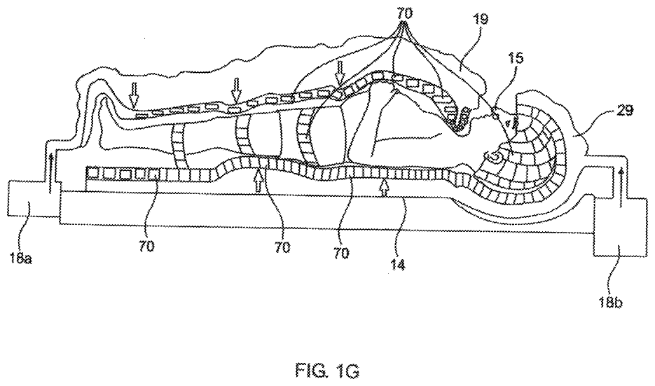

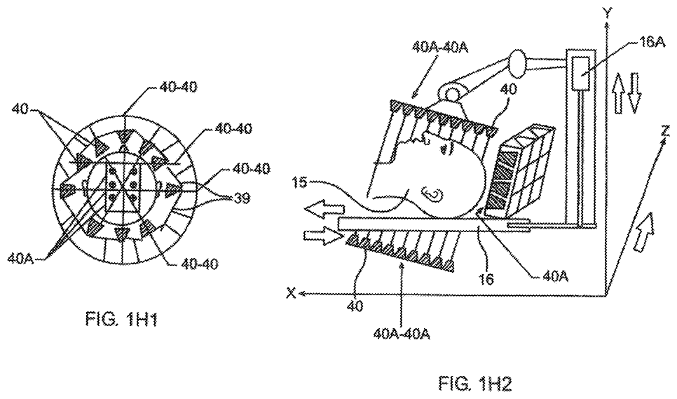

As seen from FIG. 1B, in an assembly 10 for MRI imaging, a patient 15 is resting on a subject-receiving region 20 having at least one surface 16. In one implementation, the subject-receiving portion is a scanner bore 20 and the patient is or subject is resting on a support structure 16 inside the scanner bore 20 of the assembly 10. The subject-receiving region 20 may be open or closed and may include any known version of an open MRI including one in which there is no formal bore and the subject rests on a surface so as to receive radiation from the subject's left and right sides.

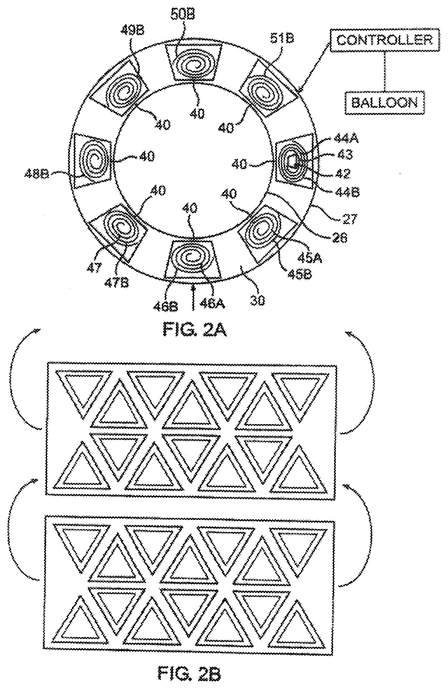

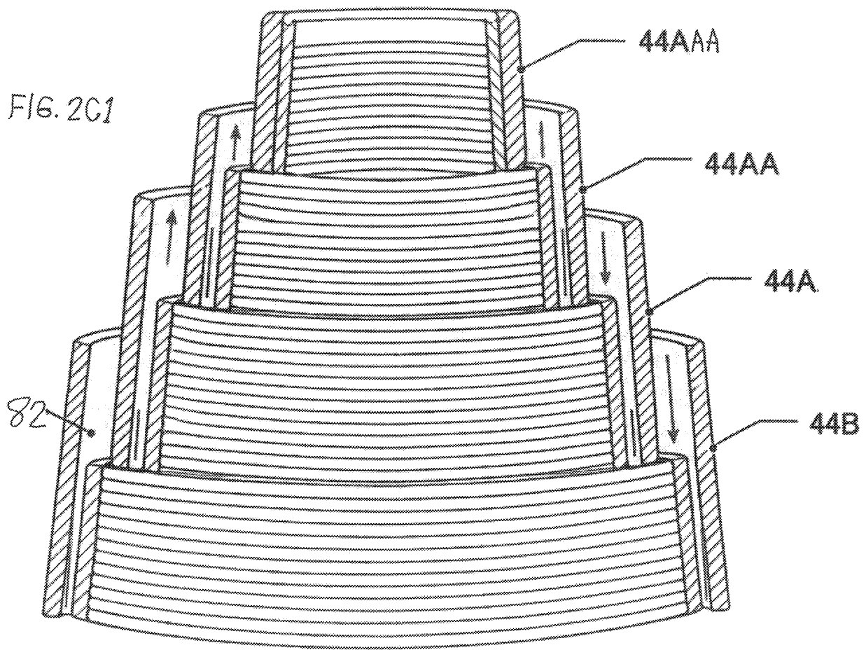

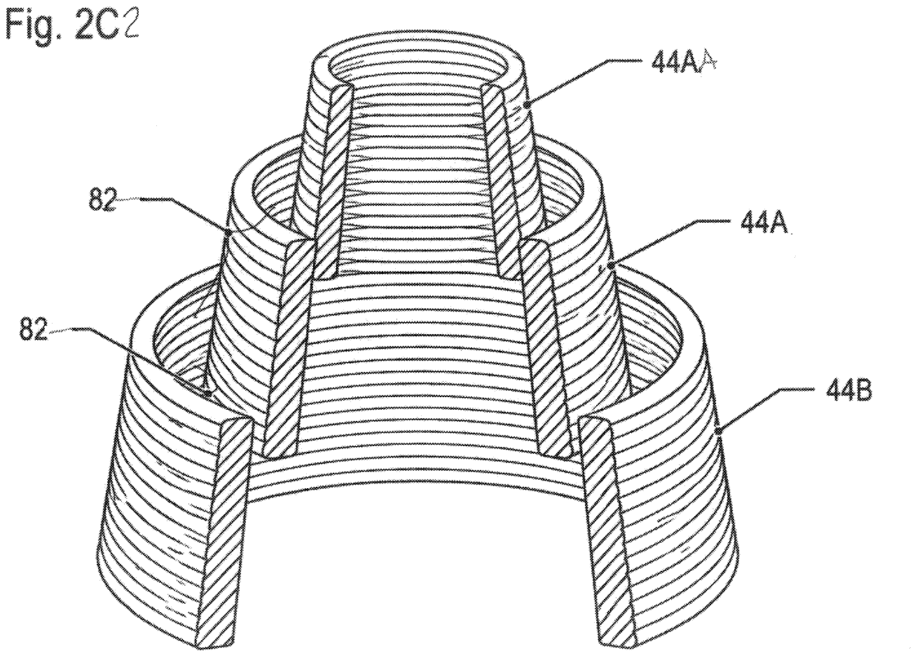

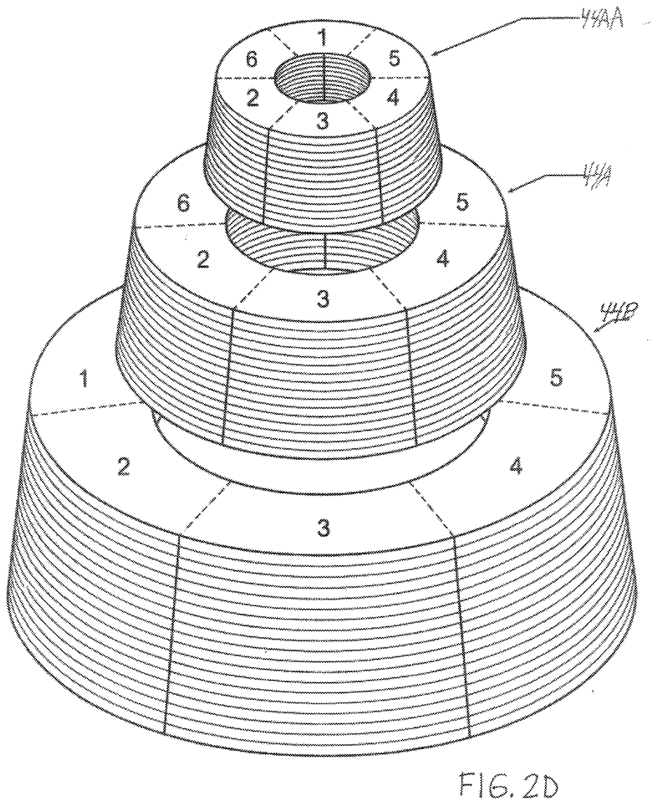

The assembly 10 may form part of an MRI imaging system in that assembly 10 interacts with a computer system 98, for example with a remote computer system 98. As seen from FIG. 1A, FIG. 1D and from FIG. 2A, the scanner bore 20 has an inner wall 26. The inner wall 26 defines an internal surface whose horizontal cross-section (wherein the “horizontal” direction is perpendicular to a longitudinal direction running parallel to the length of the subject's body) may be circular, substantially circular, curved, partially curved, substantially curved, oval, substantially oval, rectangular, substantially rectangular, or any other shape that is fully or partly curved and partly not curved or not curved.

As seen from FIG. 1A, FIG. 1B and FIG. 1C, the entire scanner bore 20 can be viewed as separate modules associated with body portions of the subject being scanned including one or more of a head module 21 (FIG. 1A), a thoracic module 22 (FIG. 1B, FIG. 1C), an abdomen module 23 (FIG. 1B, FIG. 1C), a legs module 24 (FIG. 1B, FIG. 1C) or other body portion modules such as a chest module or a pelvic module. Each module of the scanner bore 20 may comprise one or more sections of the wall of the scanner bore, each section of the one or more sections of the wall being referred to as a belt 30. Especially in the case of a tall subject, there may be more than one belt 30 for a particular module.

Although FIG. 1B and FIG. 1C depict scanner bore 20 modules as discrete and separated by a space, the length (along the length of the patient's body) of each module may be adjustable such that the scanner bore 20 depicted in these figures may lengthen so as to reduce or eliminate the gaps between the modules 21, 22, 23, etc. and thereby result in a continuous or essentially continuous wall (for example the inner wall 26) or a wall that at least appears to an outside observer to be continuous. Another option is that the wall of the scanner bore is continuous along the length of the bore 20 such that the modules 21, 22, 23, 24 are not structurally distinct but rather are defined by the distinct “volumes” of space that are scanned, (i.e. the space around the head, the space around the thorax, the space around the legs, etc.).

Inner wall 26 may be curved although not necessarily circular. Alternatively, the inner wall 26 is partly rectangular and party curved or substantially rectangular and curved, for example, at its corners. In this case, inner wall 26 may be partly curved or curved in portions of the belt 30. Alternatively, inner wall 26 is rectangular or substantially rectangular.

The term “circumferential” portion of the inner wall 26 is used herein to refer to the internal surface of the inner wall 26 regardless of whether the shape of such inner wall 26 is circular, substantially circular, curved, partially curved, substantially curved, oval, substantially oval, rectangular, substantially rectangular or any other shape that is fully or partly curved or not curved.

FIG. 2A is a schematic horizontal sectional view cut at some point along the length of the thoracic module 22 of the scanner bore 20. The viewer is looking axially as a result of the cut. FIG. 2A depicts a schematic axial view of the thickness of the belt 30. As seen in FIG. 2A, the assembly 10 may include electromagnets 40 that may be formed as a coil or coil assembly 44 and that may be configured to generate an electromagnetic field. Electromagnets 40 may be positioned on the subject-receiving surface, although this is not a requirement. For example, electromagnets 40 may for example be circumferentially along an inner wall 26, for example along an inner wall 26 of a bore 20. In certain embodiments, each electromagnet 40 comprise a metallic core 42 surrounded by a coil assembly 44. Coil assembly 44 (and other coil assemblies) may be held in place by a coil holder frame 39 (FIG. 1H1). In other embodiments, the electromagnets 40 are superconducting electromagnets and may have no core, or may have no metallic core.

FIG. 2A shows a series of electromagnets 40 shaped in the form of truncated pyramids or frustoconically shaped, according to one embodiment, attached to the belt 30 within a thickness of the belt 30.

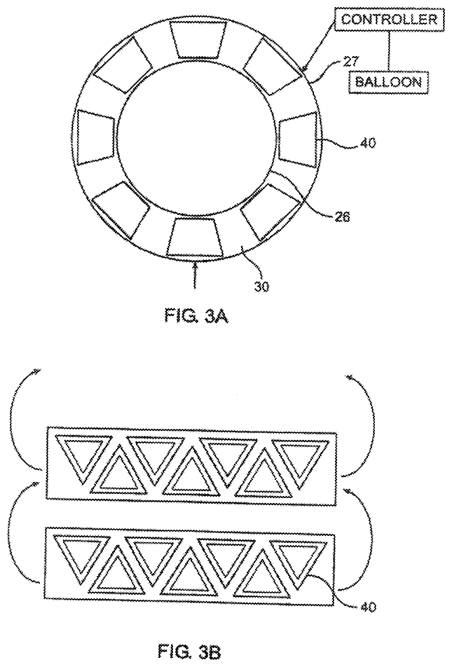

FIG. 3A is a schematic horizontal sectional view of the thoracic module looking through the belt 30 toward the space where the patient would be situated for a scan. FIG. 3A shows these electromagnets 40 in a particular non-limiting configuration designed to maximize space efficiency.

In FIG. 3B, the electromagnets 40 are shown in an alternative non-limiting configuration. In both FIG. 2B and in FIG. 3B, a first and a third (or more) of a series of the electromagnets faces in a first direction and a second (or a second and a fourth or more) of the series of electromagnets faces in a direction opposite to the first direction. Inner wall 26 may comprise portions that are considered belts 30.

As shown in FIG. 2A, each electromagnet 40 (for example one that is not a superconductive electromagnet) may include a coil assembly 44 wrapped around a core 42, such as an iron core (even though the core 42 is depicted in only one of the electromagnets 40 shown in FIG. 2A). The core's own coil 43 is attached to and integrated with the core 42, as depicted in FIG. 1A. The term “coil assembly” 44 as used in this patent application means one or more coils of a particular electromagnet 40 other than coil 43 of the core 42. In FIG. 2A each of the one or more coils which together comprise coil assembly 44 has been designated 44A (inner coil) and 44B (outer coil) (and there could be further coils more radially distant than outer coil 44B)

In some embodiments, as shown in FIG. 2C1 and FIG. 2C2, the coil assembly 44 comprises two or more inter-nested coils 44A (inner coil), second inner coil 44AA, 44B (outer coil) such that an outer coil 44B of a particular coil assembly 44 at least partially shields the electromagnetic fields generated by the inner coil 44A and second inner coil 44AA of that coil assembly 44 (and may also at least partially shield coil 43 of core 42) from coils of other coil assemblies 45, 46, 47, 48, 49, 50, 51 (FIG. 2A) that are adjacent (for example adjacent along the inner wall of the scanner bore 20 or of a particular portion of the scanner bore 20 associated with a particular module (e.g. 21, 22, 23, 24, 25 etc.) to coil assembly 44, for example coils 45A, 45AA, 45B of coil assembly 45 and coils 46A, 46AA and 46B of coil assembly 46. In FIG. 2C1, coil assembly 44 for an electromagnet 40 includes an outer coil 44B, a first inner coil 44A, a second inner coil 44AA a third inner coil 44AAA. The flowing liquid between the coils is depicted using arrows. That liquid can be water 82 in some embodiments or in can be liquid nitrogen in other embodiments (for example superconducting electromagnets). In FIG. 2C2, there a total of three coils (44B, 44A, 44AA).

Although as shown in FIG. 2A the electromagnets 40 are shaped frustoconically (truncated cones) or as truncated pyramids, and in FIG. 2C1 and FIG. 2C2 the electromagnets are shaped frustoconically, such shapes are not the only shapes that may be used. For example, in any embodiment, the shape of the electromagnets 40 may also be cylindrical, pentagonal, octagonal, etc. Accordingly, these shapes appearing in the drawings herein are not intended as any limitation for the electromagnets 40 or coil assemblies of assembly 10.

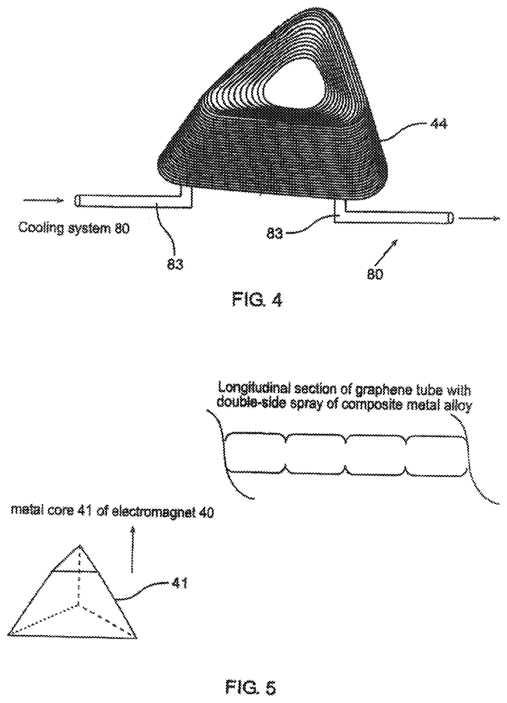

FIG. 4 also shows two electrically conducting connectors 83 configured to connect to a power supply (not shown).

In some embodiments, each of the electromagnets 40 is formed as a coil assembly and each of the coil assemblies comprise two inter-nested coils, each configured to carry pulses of electric current at a different frequency and including an outer coil 44A and a first inner coil, such that the outer coil of one particular coil assembly at least partially shields the electromagnetic fields generated by the first inner coil of the one particular coil assembly from background electromagnetic fields of coils of other adjacent coil assemblies.

In certain embodiments, the electromagnets 40 (for example each of the electromagnets 40) are formed as a coil assembly 44. Each of the coil assemblies comprise three inter-nested coils 44A, 44B, 44C, each configured to carry pulses of electric current at a different frequency and including an outer coil, a first inner coil and a second inner coil, such that the outer coil 44B of one particular coil assembly at least partially shields the electromagnetic fields generated by the first inner coil 44A and the second inner coil 44AA of the one particular coil assembly from background electromagnetic fields of coils of other adjacent coil assemblies.

The use of inter-nested coils, for example three coils including an outer coil 44B within an intermediate coil 44AA (also called second inner coil 44AA) within an inner coil 44A, generates better spatial (and temporal) resolution because each of the inter-nested coils has its own frequency of pulsation and creates its own electromagnetic field -and hence its own magnetic field gradient. Accordingly, the contrast of the subject's tissue increases. Accordingly, while certain embodiments are configured for portable assemblies for an MRI machine, the assembly 10 is also suitable for stationary MRI machines or applications.

Coils 44A (or first inner coil 44A and outer coil 44B or first inner coil 44A, second inner coil 44AA, third inner coil 44AAA (not shown), etc. and outer coil 44B) of coil assembly 44 (and of other coil assemblies 46, 48 etc.) may be formed as coiled tubes. The composition of the material that coils 44A are made from may vary. In some embodiments, the coil tubes are made from metal such as copper. In certain embodiments, the tubes are made from a combination of metal and graphene, for example 50% each. In certain embodiments, the tubes are made from a combination of metal, graphene and a composite or alloy of metals such as copper, aluminum, silver, brass and magnesium, as shown by FIG. 8A and FIG. 8B. In some implementations of this, as shown by FIG. 8C, the tubes have a variable cross-section (diameter) and may be prepared by winding a variable cross-sectional graphene material for example by hand or otherwise and then sprayed (or otherwise layered) with nanometer sized metal that may be alloys for example alloys comprising one or more of copper, aluminum, silver, brass (which is an alloy of copper and zinc) and magnesium.

In some embodiments, the number of windings of the coil 44A, 44B, etc. will not exceed 318 and will be at least about 20 or 30 or 32.

RF coils, RF circuits, and/or RF-capable segments of coil assemblies can be configured to emit a radio frequency electromagnetic field in the range of 0.1 MHz to 10 MHz. The remaining segments of the electromagnets can be configured to generate a primary, local, gradient, corrective, compensatory, and/or pre-polarizing magnetic field and are not necessarily RF emitters. The actual frequency can be adjusted by adjusting various parameters associated with the piezoelectric elements of the power supply 50, which may also affect the length of time of the pulses, using controller 60 controlling the power supply 50.

Instead of continuous electricity, the electromagnets 40 generate pulses of electric current for short periods of time such as one three-hundredth of a second to one third of a second.

In some embodiments, during any given second of time, the length of the pulse or pulses of electric current is less than 100 milliseconds. In some embodiments, during any given second of time, the length of the pulse or pulses of electric current is less than 50 milliseconds. The length of the pulses may be increased by placing capacitors between coils 44 and piezoelectric elements 52. In some embodiments, during any given second of time, there are 3 pulses per second or 333 pulses per second or in other embodiments 3000 pulses per second or 6000 pulses per second.

In some embodiments, the length of any pulse is less than half the length of the break (as defined herein) following that pulse (assuming there is a break after the pulse) and less than half the break immediately before that pulse (assuming there is a break before the pulse). In some embodiments, the length of any pulse is less than 0.1 of the length of the break following that pulse (assuming there is a break after the pulse). Without being bound by theory, the following technical details are provided as one particular non-limiting example of how to achieve the performance of the coils in some embodiments of an assembly 10 for a portable MRI:

The weight (approximate) of the coils (with the length of one face at the base 110-300 mm, at the cut top 100-270 mm and the height of 110-230 mm):
Copper wire (diameter 3-5 mm): ~5-17.6 kg
Copper tube (diameter 5-7 mm, wall thickness 1 mm): ~4-16.9 kg
Graphene tube (5mm diameter, silver-plated, 82% graphene, 18% silver)): ~2-7.25kg
YBCO (yttrium barium copper oxide) tube (diameter 7 mm, wall thickness 1mm): ~3.5-11.9 kg

To achieve a magnetic field of 0.1-1 T using the available power of a piezoelectric battery of 25-250 kW, about 32-318 turns are needed for each coil, (while the number of coils, depending on the configuration of the system, can be from 2 to 96 or more). This value is the same for all materials, since it depends on the current and the length of the coil.

Effect of ferrite cores:
Magnetic permeability (μ): Ferrite materials have a high magnetic permeability (μr), which can be hundreds or thousands of times greater than that of a vacuum (μ0) .
Magnetic field formula with ferrite: B=Lμ0·μr·N·I Where is:
   μr is the relative magnetic permeability of a ferrite core.

Effects on coil performance:
1. Increase in magnetic field: In the presence of a ferrite core, the magnetic field (B) increases proportionally μr.
2. Reduced number of turns: To achieve the same magnetic field (0.1 T), the number of turns can be significantly reduced.
3. Reduced amperage: Similarly, it is possible to reduce the amperage if the number of turns remains the same.
In other words, by using different brands and configurations of ferrites, coil configurations and materials, configurations and materials of pyropiezoelectric cells, and monitoring and control systems, it is possible to sufficiently facilitate the configuration and optimization of portable MRI.

As seen from FIG. 5, in some embodiments, each entire electromagnet 40, which includes its core 42 and its coil assembly 44, is shaped like a pyramid whose vertex has been truncated. The core alone (in embodiments in which there is a metallic core) also may be shaped substantially like a truncated pyramid. In some embodiments, each electromagnet 40 has a frustoconical shape (a truncated cone).

The core 42 of electromagnet(s) 40 produce an electromagnetic field that extends to the subject located in the scanner bore 20. This field affects the protons (or other targeted molecules/atoms such as deuterium or carbon or oxygen) in the tissue of the patient, and in particular in the water of the tissue. The affect includes changes, for example alignment, in the angle of spin of the protons, etc.

The sequence of electric pulses generated by the power supply 50 has an effect on the protons or other elements (deuterium, carbon, oxygen) being targeted while such protons or other elements are under the influence of the magnetic field. When the RF excitation component of the pulse cycle ceases, the energy signal released by the tissue of the signals, for example the energy released by the protons of Hydrogen (or by another element (in the tissue of the subject) such as deuterium being targeted) is detected by detectors 70. The assembly 10 may include detectors 70 that may be positioned circumferentially along the subject-receiving region, for example along at least one surface thereof, for example along an inner wall 26 of an open or closed scanner bore 20. The detectors 70 may be configured to detect a signal of energy released by elements in the tissue of a subject 15 located inside the scanner bore 20 at a frequency of between 0.1 MHz (100 KHz) and 10 MHz inclusive. For example, detectors 70 may be configured to detect a nuclear magnetic resonance (NMR) signal from tissue of the subject, and to transmit the NMR signal for use by a computer system in generating at least one of an image, an analysis and a diagnosis.

In some embodiments, as shown in FIG. 1E, many detectors 70 may be embedded into a cover 19 configured to be wrapped around the subject or wrapped around a body part of the subject 15 that is to be scanned in a particular module 21, 22, 23, 24, 25. In one non-limiting implementation shown in FIG. 1E, which shows only the bottom layer 19b of cover 19 with the detectors above this layer (the top layer 19a shown in FIG. 1F) MRI signal detectors 70 are embedded between each ply or layer of a two ply or two layer thickness cover 19. As shown in FIG. 1F, cover 19 may be a sheet of linen or cloth or other suitable material that is configured to cover the supine subject 15 comfortably without interfering with the breathing of the subject 15.

In some embodiments, as shown in FIG. 1F, detectors 70 in cover 19 are situated between the bottom layer 19a (for example made of rubber) of cover 19 and an air space layer under top layer 19a (for example made of linen or fabric or cloth) of cover 19 such that the act of pumping air using pump 17 into the air space by means of micro-compressor 18 (at a pressure of for example of about 0.5 atm pressure) urges the detectors 70 downward toward the body of subject 15, as seen from the pathway from micro-compressors 18 on the right side of FIG. 1F leading to the cover 19. As also seen from FIG. 1F, there is also a pathway from the micro-compressors 18 on the left side of FIG. 1F leading to the top of the mattress where additional detectors 70 are located. This pumps air into an air space of the mattress to press detectors 70 upwardly toward the body of the subject 15.

As shown in FIG. 1G, in some embodiments, a micro-compressor 18a is configured to facilitate the pumping of air into the cover 19 to press detectors 70 downward toward the subject's body and a micro-compressor 18b is configured to facilitate the pumping of air into an air space (a) in the mattress 14 to generate upward pressure on detectors 70 located below the subject 15 and (b) in a head pillow 29, so that detectors move closer to the body of subject 15.

In some embodiments, unlike in FIG. 1F, cover 19 is separate for each module 21, 22, 23, 24, 25. In some embodiments, a different cover 19 may be used for each subject 15. Alternatively, cover 19 may be sterilized for hygienic reasons by exposing the surfaces of layers 19a, 19b of cover 19 to UV radiation.

As shown in FIG. 1H1 (and FIG. 1A), the electromagnets 40 of assembly 10, in any embodiment, may be arranged such that the combination of three or more of these electromagnets 40 comprises multiple pairs or groups of electromagnets 40 (including coils) so as to generate three-dimensional images (initially). In one non-limiting implementation shown in FIG. 1H1, a pair of electromagnets 40 (called “fronto-parietal occipital” in FIG. 1H1) has an electromagnet positioned facing the frontal lobe and the second electromagnet of the pair positioned facing either the occipital lobe or the parietal lobe of the subject's brain. In a second pair of electromagnets 40, one electromagnet in the pair faces a left side of the temporal lobe and the other faces the right side of the temporal lobe. In a third pair of electromagnets 40, one electromagnet in the pair faces a left side of the frontal lobe and the other faces the right side of the frontal lobe. One, or an array 40A, of electromagnets 40 faces the parietal lobe. Another pair of electromagnets 40 (called “oblique fronto-parietal” in FIG. 1H1), comprises one electromagnet 40 that faces the left side of the frontal lobe and one electromagnet 40 that faces the right side of the parietal lobe. Due to the nonlinear, circular positioning of the electromagnets 40 in the implementation shown in FIG. 1H1, the patient 15 need not be moved by the assembly 10 in order to capture three-dimensional or multi-dimensional holographic images.

In a further non-limiting implementation shown in FIG. 1H2 the electromagnets 40 containing the electromagnets 40 including their coils are arranged in multiple, for example three arrays including an array 40A and a pair of arrays 40A-40A. Although each array of the three arrays, taken by itself, may comprise linearly arranged electromagnets 40, the electromagnets 40 in the third array 40A shown in FIG. 1H2 are not colinear or even necessarily coplanar with those of the pair of arrays 40A-40A. Moreover, in this version, the support structure 16 on which the patient 15 is resting and therefore also the patient 15 are configured to be moved by an actuator, for example by an engine 16A, through up to three degrees of freedom relative to the electromagnets 40 in the three arrays 40A. The three degrees of freedom are exemplified in FIG. 1H2 by the x-axis, the y-axis and the z-axis, along which or around which such motion occurs. Although FIG. 1H2 is depicted mainly to show stationary arrays 40A of electromagnets 40 and a movable support structure 16, the reverse is also possible (a stationary support structure 16 and movable arrays 40A, 40A-40A of electromagnets 40).

Detectors 70 may be any suitable MRI sensor for detecting aspects of the elements (e.g. Hydrogen protons, deuterium, carbon, oxygen or other elements) such as T1—T2 relaxation time, phase shift, motion (such as intracranial pulsations), the angle of the flip of the protons or other elements of the subject's tissue (or other useful aspects) that are emitted by the subject's tissue as an NMR signal when the radio frequency signal emitted by the coils 44 (or coil assembly 44) is turned off (i.e. when the RF excitation component of the pulse cycle ceases, while the primary electromagnetic field is maintained by the rapid repetition of pulses).

In some embodiments, a dedicated radio frequency (RF) coil is disposed within the coil assembly 44 and is configured to generate the RF excitation signal directed to the subject's tissue, independently of the electromagnet coils 44A, 44B that generate the primary electromagnetic field. In some other embodiments, some segments of the coil assembly can be implemented as RF-capable segments-specially configured conductive or superconducting circuits with appropriate matching, switching, resonant/capacitive elements, and the ability to transmit and/or receive radio frequency signals. In this case, the coil assembly 44, when driven by pulses of electric current at the appropriate repetition rate and tuned to the resonance frequency of the subject's tissue, generates an electromagnetic field that excites the protons at the radio frequency without a separate dedicated RF coil.

In one non-limiting example, detectors are radio frequency (RF) receivers. In some embodiments, detectors 70 comprise at least a portion of the radio frequency (RF) coils that are configured to emit a radio frequency (RF) signal to the subject. In another non-limiting example detectors 70 are NV detectors such as artificial diamonds or other materials with NV centers. NV centers in diamonds are special defects in the crystal lattice, where a carbon atom is replaced by a nitrogen atom, and one atom is missing next to it (vacancy). These centers have quantum spin states, which are very sensitive to external magnetic fields. When a diamond with NV centers is illuminated with a laser, the spin state of the NV centers changes with the magnetic field, and these changes can be read using optical fluorescence.

Detectors 70 may be very small. In this case, detectors may be a component that is separate from any RF coils. For example, NV detectors may be less than 1.0 mm, allowing them to be used in imaging applications where small size is important. Lasers may be used to excite NV centers, and fluorescence is then monitored using photodetectors. This approach allows the use of high-precision quantum effects to detect weak magnetic fields. In another version, the detectors 70 comprise at least a portion of the RF coils.

In some embodiments, associated optical excitation and fluorescence detection components may also be provided for detectors 70 or separately for assembly 10.

Detectors 70 are configured to be sensitive to a frequency of 0.1 MHz (100 KHz) to 10 MHz.

Detectors 70 may be configured to transmit the detected signal directly to a computer system, for example a remote computer system 98 (FIG. 1D) (and in other embodiments to a computer system that is local) that generates at least one of an image, an analysis and/or a diagnosis. In that case the remote computer system 98 (or local computer system 98) may include analog to digital converters. Detectors 70 may instead be configured to transmit the detected signal indirectly to a computer system 98, for example to a remote (or nonremote) computer system 98 (FIG. 1A, FIG. 1B). In this case, the detectors 70 are configured to transmit the signal to a data collection and transmission unit 88 that creates data packets that are then transmitted through to the computer system 98 in any sequence. Initially, the digitized signal in the form of data is stored on a memory storage unit of the data collection and transmission unit 88 as a data file. Data collection and transmission unit 88 may include a computer processor and software stored on memory for converting the data file into data packets for transmission in any sequence to the remote (or local) computer system. The transmission to the remote (or local) computer system for image generation and/or analysis may be implemented in a variety of manners including via communication networks (special communication networks, satellite Internet, tropospheric communication, a special cable) or even a flash drive carried by a messenger.

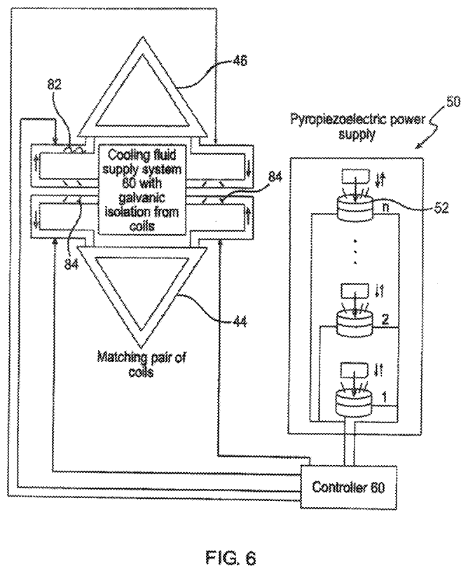

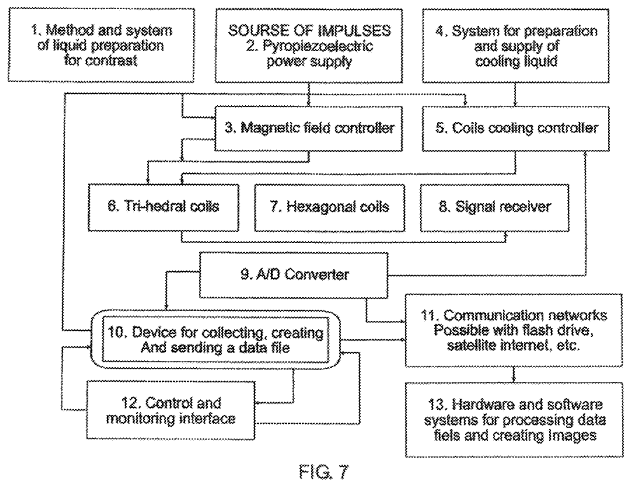

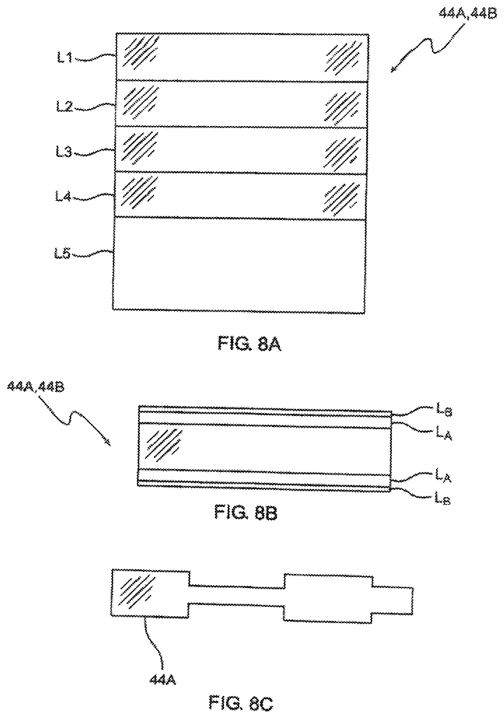

In one implementation, as shown in FIG. 6, a pyro-piezoelectric source 52 is under the control of controllers 60. A sequence of electrical pulses is generated from the piezoelectric source 52, which may be a pyro-piezoelectric source 52, and fed to coils of coil assemblies 44, 45, 46 of electromagnets 40 so as to generate a specific amount of power for each electromagnet in each module in a controlled sequence. A piezoelectric battery may feed power to electromagnetic coils with already specified characteristics and in a given sequence and does not require additional control.

In any embodiment of power supply 50, the mechanical irritation of the piezoelectric elements 52 (one non-limiting example of which are piezoelectric crystals 52) may occur at a frequency ranging from 3 times per second to 300 times per second. This is the internal resonance of the piezoelectric element. This frequency of 3-300 Hz is the frequency with which the mechanical actuator, such as a piston, is pushed by a pneumatic drive, a hydro drive or a pyrotechnic drive, and thereby mechanically affects the package (“sandwich”) of mixtures (paste, gel, powder) of piezoelectric substances 52. In one implementation, this mechanical irritation generates pulses of electric current exiting the package (“sandwich”) of mixtures (paste, gel, powder) of piezoelectric substances 52 (and directed to the one or more coil assemblies 44, 45, 46, etc.) at a particular resonance frequency anywhere from 100 KHz to 10 MHz. When the electric current exits the power supply 50 (for example the piezoelectric material 52) it is transmitted to and reaches the electromagnet 40 and its coil assemblies 44, 45, 46, etc., such that in some embodiments the frequency of the coil assembly 44 may be the particular frequency between 100 KHz and 10 MHz. In other embodiments, the frequency is adjusted higher or lower (but still within the range of 100 KHz and 10 MHz) by means of a frequency regulator or adaptor, situated between the power supply 50 (or the piezoelectric material 52) and each of the one or more coil assemblies 44, 45, 46, etc.

In some embodiments, the power generated by the electromagnetic coils of the coil assemblies 44, 45, 46, etc. ranges from 50 KWatts to 300 KWatts. In some versions, the 300 KWatts is achieved by incrementally raising the power of the pulses up to 300. In this manner the length of the breaks between pulses is of less concern (due to the superconductivity) in some embodiments In some embodiments, the frequency is increased incrementally so that the power is increased from 50 KWatts to 300 KWatts.

In some embodiments, the voltage of the electricity in the coil assemblies (44, 45, 46 etc.) ranges from 100 Volt to 10 Kilovolt (KV) with an average voltage of about 300V or 500V. The current ranges from 10 amps to 300 amps with an average current of about 333 amperes to about 500 amperes. This leads to a range of power from 1 KWatt to 3000 KWatts, with an average power of about 100 KWatts to 250 KWatts.

In certain embodiments that produce video MRI, the range of power of the assembly 10 may be between one and ten KWatts. In some embodiments, average power of from one to ten KWatts is sufficient to obtain the bulk of the magnetic resonance data. This may include operation of receiving paths, control electronics, digital processing, synchronization, image reconstruction, low-and medium-power radio frequency excitation, some gradient and correction effects, as well as multichannel data reception from coil arrays. Moreover, the generation of a dynamic image sequence can be achieved not by constant megawatt power consumption, but by multichannel data acquisition, k-space undersampling, overlapping time windows, interpolation, prediction, motion compensation, and subsequent dynamic reconstruction.

High power values, for example, up to 3000 kW, may be used as potential short-term peak values arising from the generation of short, powerful current and/or voltage pulses. In some embodiments, such pulses can be used for pre-polarization, rapid current changes in inductive coils, switching individual coil array segments, local magnetic field amplification or correction, gradient encoding, motion compensation, magnetic flux pumping, and for the coordinated operation of multiple coil arrays within a spatial array.

For these high power values of up to 3000 KWatts, such pulses can in some embodiments be generated by a distributed array of piezoelectric, ferroelectric, pyroferroelectric, capacitor, superconducting, inductive, and/or other storage-discharge energy modules. These modules can operate in conjunction with high-energy drives, including pyrothermic, pneumatic, hydraulic, mechanical, gas-dynamic, inertial, or combined drives. Energy in such a system can be pre-accumulated and then released in short pulses with a low duty cycle, allowing the peak power of individual pulses to exceed the average power consumption of the device by orders of magnitude.

For example, in some embodiments, a peak power of 3000 kW with a pulse duration of 10 microseconds corresponds to a single pulse energy of approximately 30 J, and with a pulse duration of 100 microseconds, it corresponds to approximately 300 J. With short pulse durations and a limited repetition rate, the average power of such a subsystem can remain in the kilowatt range, despite very high peak values. Therefore, the availability of pulse power up to 3000 kW does not contradict the fact that the main part of the video MRI information mode can be performed at an average power of up to 10 kW.

In certain embodiments, further reduction in continuous power requirements can be achieved by using cryogenic operation of coils and magnetic elements, superconducting or high-temperature superconducting circuits, permanent magnets, cryogenically cooled conductors, and magnetic flux pumping devices, including flux pumps. In superconducting mode, energy can be stored in the magnetic field of the circuit with minimal ohmic losses, and the flux pump can provide current injection or maintenance in the superconducting circuit without the need for continuous high-current injection through massive current feedthroughs.

Coils of coil assemblies 44, 45, 46 may be made of a copper tube (and optionally also of aluminum, magnesium, silver, or a special alloy), the tube having a special variable cross-section. Inside the tube is a special cooling liquid-for example water or hydrogenated water in some cases including a suspension of nanoparticles fed from a nanoparticle storing source or a nanoparticle generating system. The cooling liquid 82 circulates under a certain pressure and at a certain speed.

Using coils of various configurations under the control of controller 60 and as a result of feeding a special sequence of pulses in a scanned volume within the scanning bore 20, there is created a multitude of small individual scanned volumes (along a given trajectory) whose sizes vary from 1 mm3 to 1 cm3.

As seen in FIG. 7, the received data are collected in data acquisition units or data collection units 88 (FIG. 7 blocks 8, 9, 10) which are configured to transmit this data (for example in some versions after creating data packets) through communication networks or systems 11 to block 13, which is the computer system in FIG. 7, for example a remote computer system. The data acquisition and collection units 88 may include a receiver (FIG. 7 block 8) to receive the signal from detectors 70 (i.e. a radio frequency signal) and an analog digital converter (FIG. 7 block 9) and memory storage as well as software and hardware (FIG. 7 block 10) for collecting, creating and sending data files.

The coils 44, 45, 46 etc. may be sprayed with metal such as silver (or gold) in order to improve the ability of the coils to be cooled. The sprayed layer of metal may be external and/or internal

The coils 44, 45, 46 may be mounted in an inflatable frame, which minimizes the time of preparation of the system for operation and ensure its mobility.

Regarding the strength of the electromagnets 40 in assembly 10, there are several ways of implementing this. In one implementation, the strength of the electromagnets 40 is boosted immediately to 1.6 T (Tesla) (for example by adjusting the electric current), and as a result the strength of the NMR signal produced by the subject's tissue and received by the detectors 70 is correspondingly increased. In certain embodiments, the peak field of 1.6 T serves as a pre-polarization field, after which the field is reduced to the operating level at which RF excitation occurs at the resonance frequency of the subject's tissue within the 0.1-10 MHz range. In some embodiments, oxygen-17 or another element in the tissue of the subject is the primary target, for example at higher field strengths.

The strength of the electromagnets 40 is high and the image quality is good. There is a relatively high signal to noise ratio with the resulting imaging having high contrast and spatial resolution. This can be further enhanced by adding nanoparticles into the water of the cooling system or by using hydrogenated water.

In another implementation regarding the strength of the electromagnets 40 in assembly 10, one starts with a Tesla strength of 50 milli Tesla and gradually increases the strength of the electromagnets 40 (and hence the strength of the signal) to a strength of 1.6 T, for example by increasing the current.

Increasing the strength of the electromagnet can be accomplished in one or both of the following two ways. One way is simply to increase the current. A second way is to use to increase the piezoelectric effect, for example by having the actuator contact (for example rub or deform) the piezoelectric material with greater force or frequency. In one version, the actuator exerts pressure on plates that sandwich piezoelectric crystals.

Either way the result is the same-a relatively high signal to noise ratio with the resulting imaging having high contrast and spatial resolution. This can be further enhanced by adding nanoparticles into the water 82 of the cooling system 80 or by using hydrogenated water.

Assembly 10 may also include a built-in power supply 50. Power supply 50 may be connected to the electromagnets 40 and may be configured to generate pulses of electric current. The power supply 50 may comprise piezoelectric material 52 and may comprise an actuator 54. The power supply 50 may be controlled by a controller 60 so as to generate the pulses of electric current at a repetition rate and for a duration specified by the controller 60.

For example, power supply 50 may be connected to each of the electromagnets 40. In one implementation, power supply 50 comprises piezoelectric materials such as pyroelectric piezoelectric material 52, for example solid materials, powders or creams. The piezoelectric material may be pyroelectric piezoelectric material. The material 52 may comprise crystals and/or ceramic or other solids including but limited to powders or viscous creams. The power supply 50 may also include actuators 54 that generate heat stress for example as a result of a mechanical action (such as pressing or rubbing or deforming) so as to affect or deform the pyroelectric material 52. In the case of piezoelectric material 52 that is not pyroelectric material, the actuator(s) 54 may be a mechanical actuator such as a movable plate or a pneumatic or a hydraulic actuator and may include plates that sandwich the piezoelectric material 52.

The power supply 50 may be configured to generate pulses of electric current and an electromagnetic field. In some embodiments, the power supply 50 comprises a separate element of piezoelectric material for each of the electromagnets 40.

One or more of a frequency, a duration, a power and a polarization of the pulses of the electric current transmitted to each of the electromagnets 40 is independently determined (in some embodiments using artificial intelligence) by virtue of being connected to the power supply 50 being connected to and/or controlled by computer controller 60.

In some embodiments, each of the electromagnets 40 in combination with the responsive detectors 70 may be considered as an independent scanning system.

Power supply 50 may be configured, based on controller 60, to determine a frequency of the pulses of the electric current transmitted to each coil assembly 44. In some implementations, the frequency of the pulses of the electric current transmitted to each coil assembly 44 is independently determined such as that different coil assemblies, and therefore different electromagnets 40, may have different pulse frequencies. This could be efficient where different modules 21, 22, 23, 24 of the scanner bore 20 are geared for different body parts and therefore require different frequencies (for example if the resonance frequencies of the body parts are different due to having different water concentrations).

Power supply 50 may be controlled by a controller 60 so as to generate the pulses of electric current at a specified repetition rate and for a specified duration. Controller 60 may control an actuator. The actuator may be one or more of a pneumatic actuator, a hydraulic actuator and a chemical actuator. The actuator may be a mechanical actuator that exerts pressure on the piezoelectric crystals or piezoelectric material. The actuator may for example exert pressure on plates that sandwich the crystals or piezoelectric material.

Such an actuator may be controlled by a processor and therefore the repetition rate and duration of the pulses of the electromagnetic coils 44 may be controlled. There are several non-limiting examples of how to accomplish this. For example, regulating the frequency of the magnetic field coils and other necessary parameters (amplitude, power and pulse duration, etc.) may be accomplished by controlling the power, speed, frequency, duration (quantity) and combination of all together of the pneumatic drive for mechanical action on the piezoelectric element or elements 52. Alternatively, combining the composition and quantity of piezoelectric mixtures. Alternatively, controlling the power, speed, frequency, duration (quantity) and combination of all together of a hydraulic drive for mechanical action on the piezoelectric element(s). Alternatively, controlling the power, duration, time and sequence of mechanical action on the piezoelectric element (elements) using pyrotechnic cartridges (supply of charges from a horn, box, tape, drum) due to partial removal of powder gases from the working cylinder (where the piston moves, acting on the piezoelectric element) or using a mechanism for feeding pyrotechnic cartridges similar to a “toothbrush” type mechanism for faster action. Or using a mixture of liquid or gaseous fuel and atmospheric air to set in motion and control the piston acting on the piezoelectric mixture.

The frequency of the electricity exiting the piezoelectric elements 52 and the frequency of the electromagnetic coils in the one or more coil assemblies 44, 45, 46, etc. will either be identical or, if a frequency regulator is inserted between the piezoelectric mixture 52 and the coils 44, the frequency of the coils in the one or more coil assemblies 44, 45, 46, etc. will be higher or lower (but still within the range of the 100 KHz and 10 MHz).

One non-limiting implementation of a system or method for controlling pneumatic, hydraulic, pyroelectric and/or thermal drives is mechanical control systems with control programs in a form similar to a drum-cam system (for rough settings) and their combinations with electronic control systems for fine tuning of electric current characteristics.

In one non-limiting implementation of this, a drum or disk with cams mounted on the surface, which may be driven by a spring mechanism, similar to a clock, can rotate to activate various elements of the system in a given sequence, i.e. each cam closes or opens electrical contacts or pneumatic-hydraulic valves, or turns on or off the mechanism for feeding pyropatrons, or turns on or off the valves and contacts that control the operation of the heat engine (where the same piston moves, mechanically acting on the piezoelectric elements). Such a design is configured to reproduce complex programs specified physically with sufficient accuracy, and changing control programs is simple: it is enough to replace the disk, drum or tape with cams with another one and with a different program. By changing the size of the cams (height), their shape, the distance between them, the number of simultaneously read tracks with cams, such a drum, disk or tape can be adjusted to control the frequency, duration and amplitude of the pulses supplied to the drives (pneumatic, hydraulic, pyro-and thermal drives).

In a further non-limiting implementation, cycle cams may be configured to create a sequence of operations whereby different actuators and contacts are activated or deactivated at precisely defined intervals. This allows complex cycles of action on piezo elements 52 to be set up. This will also allow a single specific program to be repeated many times, which is suitable for setting pulse parameters such as frequency and duration at specified intervals.

In a further non-limiting implementation, Stepping drums with switches that are activated at each step. Such a drum can sequentially close and open electrical circuits, setting a program for each actuator. This allows for flexible control with more complex programs, since each step can correspond to a unique configuration, which is suitable for situations where a precise sequence is required, such as changing polarization or phase.

In a further non-limiting implementation, notched thumbwheel switches may be used in which a disc (mechanical, hydraulic or pneumatic) with notches on its surface rotates and each notch opens or closes a circuit which activates an actuator (pneumatic, hydro, pyro and thermal actuators). Controlling the rotation speed of the disc allows changing the duration and frequency of the pulses, which is suitable for systems where periodic repetition of pulses with the ability to be adjusted is required. This can be used to adjust the duration of the phases and the frequency of pulses in the case of sequential action on piezoelectric elements. In another non-limiting implementation, mechanical timers with movable cams move (adjustable) cams are configured to activate actuators in a given sequence and at different intervals. This allows setting complex time programs with the ability to be adjusted, which is well-suited for adjusting both the duration and repetition rate of pulses.

Power supply 50 may generate the electricity and the EMF field for short periods of time such as 3.3 milliseconds (one three hundredth of a second) up to 333 milliseconds (one-third of a second) or anything in between such as 32 milliseconds plus or minus 25%, or 3-50 milliseconds or 3-100 milliseconds or 20-40 milliseconds or 250 milliseconds or less or 333 milliseconds or less or 100-250 milliseconds or less than one second or 10-50 milliseconds.

In some embodiments, the number of pulses per second of electricity is 3 to 300. In embodiments in which the generated images are needed in the form of video, the number of piezoelectric elements used is increased (for example up to 20 such piezoelectric elements) (such as piezoelectric crystals) and as a result the number of pulses per second may be up to 3000, or in some embodiments, the number of pulses per second is 6000.

In some embodiments, the number of pulses generated per second is 30 or fewer In some embodiments, the number of pulses generated per second is three (or 3 or fewer). In some embodiments, the number of pulses per second is four (or 4 or fewer). In some embodiments, the number of pulses per second is two.

The power supply 50 may be implemented as a distributed pulsed power system configured in some embodiments to generate currents from 10 to 399 amperes. The power supply 50 may be configured in some embodiments to generate a voltage of 100V to 10 kilovolts. The power supply 50 may be configured to in some embodiments generate 1 to 3000 kilowatts of peak, pulse, short-term, average and/or distributed power.

Power supply 50 may include an adaptor 51 that has a rechargeable battery (an accumulator) and/or capacitors so as to connect the power to coils from external vehicles and external stations. The adaptor 51 may be used to connect to various types of equipment, for example, electrical equipment in a car, an armored personnel carrier, an aircraft, a recue drone, a land vehicle such as an ambulance or an armored personnel carrier, a spacecraft, a space station or other known electrical networks.

In certain embodiments, especially but not exclusively applicable to an assembly 10 that is configured to output video MRI, each coil assembly 44 may be implemented as a multilayer electromagnetic module containing three to five or more nested coil layers (for example coil assembly 44 having for example four inner coils 44A, 44AA, 44AAA, 44AAAA (not shown), and outer coil 44B), where each coil layer can be divided into multiple independently or in groups of controllable segments, preferably from three to 12 segments (for example in FIG. 2D there are six segments), forming a spatially distributed structure for excitation, shaping, correction, gradient control, local focusing, compensation, and/or shielding of magnetic and electromagnetic fields.

FIG. 2D is a schematic view showing three inter-nested coils 44 of a coil assembly 44 including an outer coil 44B having the widest diameter, a first inner coil 44A having a smaller diameter and a second inner coil 44AA having the smallest diameter of the three (the differentials in diameter is to allow inter-nesting). These coils each have six segments, which is merely an example of the number of segments. FIG. 2D depicts the three coils (44B, 44A, 44AA) similar to an exploded presentation, though they may be inter-nested within one another.

The inner coil layers (2-4 inner coil layers from amongst 44A, 44AA, 44AAA, 44AAAA) and their segments can differ from each other in geometry, number of turns, conductor cross-section, conductor material, electrical resistance, inductance, operating current, frequency range, thermal load, Q factor, cooling method, winding direction, electrical connection diagram, phase, polarity, and excitation algorithm. These parameters are selected to enable selective activation of a single segment, a group of segments, a single coil layer, several coil layers, or all coil layers and segments of a coil array, depending on the selected operating mode, the region of interest, the required penetration depth, spatial resolution, image reconstruction speed, permissible thermal load, and the required magnetic field configuration.

In any embodiment herein, notwithstanding the examples that are depicted in the drawings, the windings of the coils 44 (whether inner coils or outer coils) may be in any direction or angle of direction and not only horizontal or vertical directions but also oblique or any other angle of direction or combinations thereof.

Segments and layers of a coil array can be excited sequentially, in parallel, in pairs, in groups, phased, pulsed, quasi-continuously, combinatorially, according to preset programs, adaptive feedback algorithms, or in a mode of dynamic current redistribution between segments and layers. This design allows for the generation of not only a basic magnetic field but also local corrective, gradient, compensating, focusing, and shielding fields, as well as the ability to change the spatial configuration of the field in real time depending on the movement of the object being studied, respiratory wave, cardiac pulsation, blood flow, lymph flow, interstitial fluid, and other dynamic processes.

Optionally, the coil assembly (44, 45, etc.) may additionally contain permanent magnets, magnetically conductive elements, ferromagnetic, ferrite, diamagnetic, paramagnetic, superconducting, and/or high-temperature superconducting elements designed to amplify, stabilize, direct, concentrate, compensate, or shield the magnetic field. In certain embodiments, these elements can operate in conjunction with coil layers in cryogenic mode, including high-temperature superconductivity, for example, when cooled with liquid nitrogen, with the ability to form stable current circuits, reduce electrical losses, and increase the efficiency of magnetic field generation.

The outer coil layer (for example 44B, 45B, etc.) of a specific coil assembly (for example 44, 45, 46, etc.) can function, at least in part, as a power, correction, compensation, and/or shielding layer, reducing the effects of external electromagnetic interference, parasitic fields from adjacent coil assemblies, reverse induced currents, cross-coupling, and background electromagnetic noise. Moreover, the outer layer can be implemented not only as a passive shield, but also as an active, controlled element, generating antiphase, compensating, or matching fields to improve the operational accuracy of the inner coil layers.

With respect to the coils, electrical insulation, thermal stabilization, and mechanical fixation of turns, segments, and interlayer spaces can be achieved by filling the space between turns, layers, and segments with a dielectric, heat-resistant, and/or thermally conductive material, including corundum, aluminum oxide, ceramics, glass ceramics, Teflon, fluoropolymer, polyimide, epoxy composite, ceramic composite, or other material with similar electrical, thermal, mechanical, and cryogenic properties. This material can simultaneously function as an electrical insulator, a thermally conductive filler, a structural frame, a vibration-resistant retainer, and a means of preventing interturn breakdowns.

Assembly 10 is configured to output a video image including at least one full MRI protocol of a whole body of the subject and the duration of the video image may be from 60 to 90 seconds. In the case of a video image covering at least one full MRI protocol of an organ or a region of the subject, the duration of the video image may be 15-30 seconds and may not exceed 30 seconds.

As shown in FIG. 4 and in FIG. 6, assembly 10 may also include a cooling system 80. In one implementation of the coils 44, the coils are tubular and comprise metal tubes (or graphene and metal) within which a cooling fluid runs. The cooling system 80 utilizes water 82 or another liquid (for example liquid nitrogen) running through these tubes.

Especially but not exclusively for an assembly 10 that produces a video MRI, cooling of the coil assembly can be provided by a distributed system of channels, cavities, microchannels, capillaries, tubular sections, or interlayer passages located within the conductors, within the windings, between the windings, between coil layers, and/or in the supporting body of the assembly. Liquid nitrogen, another cryogenic coolant, a dielectric coolant, a heat transfer fluid, air, gaseous or liquid carbon dioxide, or combinations thereof can be pumped through these channels. In a stationary or high-field version, liquid nitrogen cooling may be preferred to ensure high-temperature superconductivity, while in a portable version intended for autonomous delivery, including by unmanned vehicles.

In certain embodiments, as seen in FIG. 6, the efficiency of the cooling system 80 is enhanced by including a series of projections 84 that extend from an inner wall 26 of the tubes so as to induce a water vortex in a flow of the water 82. The water vortex increases an efficiency of the cooling system 80.

Nanoparticles 86, such as metal nanoparticles 86, have in some embodiments been added to the cooling fluid 82 (for example in the water 82) to increase a signal to noise ratio for the transmitted signal.

In one embodiment, the coils of coil assembly 44 are made in part (or completely) from graphene. Instead of fully metallic coils or tubes, one option is to use a combination of metal and graphene or a combination of metal and graphene and a particular ceramic to dramatically reduce the weight. This reduces the weight in two respects-one in that the graphene portion weighs less than metal (graphene is one sixth the weight of copper for example) and further that the use of graphene which has a high tensile strength allows for use of a thinner diameter “metallic” tubular coils. In some embodiments, this achieves a drastic weight reduction of the entire assembly to the point of weighing only seven (7) Kgs. It is noted that graphene acts like metal in that it can have greater electrical conductivity than copper and in that it has superior charge carrier mobility. Graphene is considered a quasi-metal. Furthermore, manufacturing technology currently exists (for example creating composites by mixing graphene with raw metal powder and then compacting into the desired shape) to integrate these two components so as to form an alloy of metal and graphene (for example 20% metal and 80% graphene).

For MRI assemblies described herein in any version, whether portable or stationary, the percentage of metal in the mixture of metal and graphene used for the electromagnetic coils or coil assemblies 44 ranges from as low as 2% metal (and 98% graphene) to as high as 30% metal (and 70% graphene).

In one implementation of the coils for the electromagnets 40 of the assembly 10, which may be portable or stationary, as shown in the partial longitudinal cross-sectional view of FIG. 8A (showing for example a top half of the thickness of the tube/coil) each coil 44A, 44B, etc. in coil assembly 44 comprises several layers—L₁, L₂, L₃, L₄. For example, an outermost layer, L₁, is made of metal (for example in the form of a powder), the adjacent layer, L₂, is made of graphene (for example in the form of a powder), the next layer, L₃, is made of a particular ceramic (for example in the form of a powder) and the innermost layer is made of metal (for example in the form of a powder).

In one non-limiting version, electromagnet 40 including core 42 and the layers of the coils of coil assembly 44 are manufactured using 3-D printing. 3-D printing is used to manufacture these layers of the coils of the coil assembly 44 and form the tubular coil. The powder layers are then heated in an oven at for example about 900 degrees Celsius and left to cool. The result is a coil tube. In FIG. 8A, the cavity of the coil 44A is denoted by reference numeral L₅. As noted, this cavity L₅ may contain a fluid, for example liquid nitrogen.

In certain embodiments, coils 44A, 44B, etc. of coil assembly 44 of electromagnets 40 are made of a particular ceramic called yttrium barium copper oxide, whose preparation is known. This ceramic achieves superconductivity at lower temperatures than typical superconducting magnets that use liquid helium. Furthermore, this ceramic is far lighter than copper and even lighter than graphene.

As shown in the longitudinal sectional view of FIG. 8B, moreover, in some embodiments the yttrium barium copper oxide ceramic layer L_{A} is insulated by an outer layer L_{B}. The outer layer may be aluminum oxide (Al₂O₃) (for example corundum) or silicon carbide or silicon nitride or Teflon® or any of these with alternating layers of silicon dioxide. In certain embodiments, this insulated ceramic material is produced using 3-D printing. In some embodiments, the 3-D printing also places liquid nitrogen (or liquid nitrogen together with a smaller quantity of hydrogen) in the coil tubes. The preparation process may include steps in which after the insulation layer L_{B} is added to the yttrium barium copper oxide material layer L_{A} the combined material is heated in an oven, for example at approximately 300 °C for approximately 24 hours, and then removed and allowed to slow cool, for example for approximately 24 hours. The result is a material that is not brittle, unlike prior art superconducting ceramics, which are very brittle. Furthermore, the material is very lightweight.

The coils 44A, 44AA, etc., 44B are part of a coil assembly 44 that may contain as many as 8 inter-nested coils. Each nested coil 44A, 44AA (FIGS. 2A), 44AAA (not shown), etc., 44B is made of the above multiple layers. Typically the coolant (water or liquid nitrogen) is within the tubular coils of the one or more coil assemblies but optionally, if the coolant is liquid nitrogen, this liquid nitrogen may fill the space between the inter-nested coils 44A, 44AA (FIGS. 2A), 44AAA (not shown), etc., 44B etc. The liquid nitrogen is cooled to a temperature than is hotter than minus 200 °C, in some embodiments, hotter than minus 185 °C. The liquid nitrogen is cooled to a temperature of about minus 183 °C in some embodiments. The liquid nitrogen is configured to cool the metal (for example copper), the ceramic and the graphene to be approximately the same temperature as the liquid nitrogen. Applicant has discovered that the introduction of the liquid nitrogen at approximately minus 183 °C (plus or minus 7 degrees C) will form a superconductive effect. Without being bound by theory, this should cause the electrical resistance of the ceramic to go down towards zero- and hence the electrical resistance of the metal and graphene will likewise decline to or towards zero. Importantly, this superconductive effect may be achieved by using a fluid cooled to only about 183 °C as opposed to a fluid cooled all the way down to 270° Celsius such as by using liquid helium. This lowers the cost of manufacturing considerably, in only due to the very expensive nature of liquid helium.

In some versions of method 200, there is a step of inserting liquid nitrogen in a cavity of the tubular coil, wherein the liquid nitrogen is hermetically sealed.

The remote (or nonremote) portion of the system not shown in the drawing includes the computer system that receives the transmitted signal from the data collection unit 88 or from detectors 70 and creates an image and analyzes the image. This computer system may use specialized software for MRI. Such specialized software exists for processing and analyzing MRI signal data to generate images, analyses and diagnoses, including for example OsiriX (Pixmeo SARL), FSL (FMRIB Software Library, University of Oxford), and Nordic Neuro Lab software. Such software is commercially available and its use by a person of ordinary skill in the art requires no undue experimentation.

For example, Nordic Neuro Lab offers specialized software for MRI data analysis, including functional MRI (fMRI) and image processing. This specialized software is often used in research and clinical settings. In addition, the following specialized software, called OsiriX is a program for processing and analyzing MRI and CT images. It is widely used by radiologists and researchers to view and process medical images—it can be used to process and generate the images from the signal transmitted to the computer system, for example a remote computer system, in the embodiments described herein for the portable MRI imaging assembly (as well as for a stationary MRI imaging assembly). In addition, MRI Research Facility Software—available as a University of Iowa MRI Image Analysis Software Toolkit—can be used for analysis of the images. This software includes FSL, AFNI, Slicer3 and other instruments designed for research purposes.

In some embodiments, scanner bore 20 is radially or circumferentially adjustable. As shown in FIG. 2A and FIG. 3A, along the inner wall 26, there may be spaces along the band between the electromagnets or between the detectors. A series of actuators, for example pneumatic actuators, may be situated between the electromagnets 40, or between the coil assemblies 44 of the electromagnets 40, in order to effectuate an adjustment of a diameter or circumference of the scanner bore 20. In some embodiments, electromechanical actuators are situated between coils of the electromagnets so as to precisely position each coil of the coil assembly 44 of the electromagnet 40. In some versions, the positioning is configured to take into account a breathing, heartbeat and/or unconscious micromovements of the subject. In one implementation shown in FIG. 1D, assembly 10 includes a central electromechanical actuator (such as a gear 94) from which multiple connecting levers 95 extend to portions of the wall 20A of the scanner bore 20 for precise adjustment of the diameter or circumference of the scanner bore 20. Wall 20A includes the different portions thereof (for example wall portion 21A of head module 21) in the event that the various modules 21, 22, 23, 24, 25, etc. are spaced apart from one another.

Assembly 10 may include an adjustment mechanism for the scanner bore 20 or for particular modules 21, 22, 23, 24 etc. of the scanner bore 20. In one implementation, assembly 10 has at least one of:
(a) an adjustment mechanism for a radial adjustment of the scanner bore comprising a series of pneumatic actuators situated along an inner wall of the scanner bore (for example situated between electromagnets 40); and
(b) a precision adjustment mechanism for precise tuning or subsequent fine tuning of the radial or circumferential adjustment of the scanner bore 20 comprising a series of electromechanical actuators situated along the inner wall 26 of the scanner bore 20, wherein a diameter of the scanner bore is configured to be adjusted by the one or more adjustment mechanisms.

The electromechanical actuators are situated between the electromagnets coils 44A, 44B, etc. so as to precisely position each coil 44A, 44B, etc. of the coil assembly 44, taking into account the patient's breathing, heartbeat and unconscious micromovements.

Inner wall 26 of the scanner bore 20 may have circumferentially or radially collapsible sections between the electromagnets pursuant to the adjustment mechanism.

The scanner bore 20 may comprise multiple modules 21, 22, 23, 24 that are each independently radially adjustable. The multiple modules may include at least two or at least three of or all four of (i) a head module 21, (ii) a thorax module 22, (iii) an abdominal module 23, (iv) chest module and (v) a leg module 24. Each of the multiple modules may be independently radially adjustable as a result of having one or both of the series of pneumatic actuators and the series of electromechanical actuators.

In some embodiments, the head module 21 has from 32 to 36 of the electromagnets. The quantity of the electromagnets in the assembly 10 is a number from 16 to 256 and each of the electromagnets comprises a coil assembly.

As seen from FIG. 2B and FIG. 3B, in one particular non-limiting embodiment, a first and a third of a series of the electromagnets faces in a first direction and a second of the series of electromagnets faces in a direction opposite to the first direction.

One or more of a repetition rate, a duration, a power and a polarization of the pulses of the electric current transmitted to each of the electromagnets is independently determined by virtue of being connected to the computer controller 60.

One embodiment of assembly 10 is a portable assembly for an MRI imaging system that includes:
a scanner bore having an inner wall;
electromagnets positioned along the inner wall, each electromagnet comprising a metallic core surrounded by a coil assembly;
each of the coils comprise metallic tubes, the assembly further comprising a cooling system that includes water running through the tubes for cooling the electromagnets;
detectors positioned along the inner wall and configured to detect a signal of an electromagnetic field and to transmit the signal to a remote computer system for generating one or more of an image and an analysis,
wherein at least one of the following is true:
   (i) the water is hydrogenated water;
   (ii) the water contains suspended nanoparticles; and
   (iii) projections extend from an inner wall of the tubes to induce a water vortex.

The projections may extend from the inner wall of the metallic tubes at an acute angle to the inner wall in relation to a flow of the water and are shaped to induce the water vortex to increase an efficiency of the cooling system.

As shown by FIG. 8C, the metallic tubes of coils or coil assemblies 44 may have a variable cross-section so as to induce the water vortex in a flow of the water, the water vortex configured to increase an efficiency of the cooling system.

In any embodiment herein, the coils or coil assemblies 44 of each of the electromagnets 40 may optionally be immersed in a heat-absorbing mixture (e.g., a salt mixture) so as to increase a heat capacity of the immersed electromagnet, for example by twenty or more times that of water.

As shown in FIG. 1C, another embodiment of the assembly 10 is a portable assembly for an MRI imaging system, the assembly 10 configured to accommodate human adults and children. The assembly 10 shown in FIG. 1C depicts a scanner bore 20 divided into multiple (two or more) modules from among (i) a head module 21, (ii) a thoracic module 22, (iii) an abdomen module 23 and (iv) a leg module 24. Each of the multiple modules may include electromagnets 40 situated along an inner wall 26 of the module. Assembly 10 may also include a power supply 50 controlled by a controller so as to generate pulses of electric current at a specified repetition rate and for a specified duration and detectors 70 positioned along the inner wall and configured to detect a signal of an electromagnetic field from the electric current and to transmit the signal to a remote computer system for analysis. Power supply 50 may be inside base 93. In that case, there would be apertures (not shown) that allow thin cable to connect from power supply 50 to the electromagnets 40 and their coil assemblies 44.

Each of the modules is configured to independently perform an MRI scan. The scanning by the head module 21 of the subject's head may be performed without regard to whether another module of assembly 10 may be scanning the subject's legs, thorax or abdomen, for example, and so on for all of the modules in assembly 10. Moreover, each of the modules is also configured to simultaneously perform an MRI scan while one or more other modules of assembly 10 performs an MRI scan. Each of the modules of assembly 10 is also configured-once the scan occurs-to transmit data regarding the results of the MRI scan to the remote computer system 98 for analysis.

Furthermore, the adjustment feature of the bore modules 21, 22, 23, 24 (and module 25 or other modules in certain embodiments), wherein the module is for example radially adjustable or circumferentially adjustable, is shown in FIG. 1D with respect to head module 21. It is also noted that in some embodiments the adjustment of a portion of the bore 20 (and hence the diameter or volume of the space inside the module) of the modules of assembly 10 is independent of the adjustment of one or more of the other modules of assembly 10.

FIG. 1D shows one particular adjustment mechanism 90 for electromechanical adjustment of any of the modules of the assembly 10. In this mechanism 90, the portion of the scanner bore 20 that is part of head module 21 can be implemented as an expandable helmet 21A. In some embodiments, as shown in FIG. 1B, there may be one or more soft pillow elements made of soft material 29, or filled with air between the subject's head and the inner wall 26 of the helmet 21A (which is the inner wall 26 of the head module 21. Air compressor 29A (FIG. 1B may be used to control the volume of air in these pillows 29. This is to fixate the subject's head.

As shown in FIG. 1D, the helmet 21A is surrounded by a helmet holder 21B that may sit on a base, which may be a general control box that may incorporate the power supply 50. FIG. 1D depicts an adjustment mechanism 90 for radially or circumferentially adjusting the size of a head module 21. This adjustment mechanism 90 is equally applicable to other modules 22, 23, 24, 25, etc. that may form part of assembly 10. Adjustment mechanism 90 is specifically configured to adjust the diameter or volume inside the scanner bore of a module of assembly 10, for example of head module 21.

As shown in FIG. 1D, adjustment mechanism 90 may be electromechanical. The electromechanical adjustment mechanism 90 shown in FIG. 1D may include one or more micro engines 91, which may be situated in any location from which they can effectuate their purpose as described below. The micro engines 91, or an on/off switch 92 that controls the micro engines 91 may be in an outer portion of the helmet 21A. An on/off switch 92 for the micro engines may be positioned in a base 93 of the head module 21, although this is one example of where such a switch can be.

As shown in FIG. 1D, micro engines 91 are configured to move, for example to turn, one or more gears 94 that are integrated with levers 95 whose distal end may face in a direction toward the subject being scanned, or in particular the subject's head 17. The distal end of each of levers 95 may penetrate and attach to the wall 20A of the bore 20 of head module 21. Wall 20A includes inner wall 26 and outer wall 27. The turning of gears 94 in one particular direction pulls levers 95 and exerts a force pulling wall 20A outward. As a result of the accordion-like shape of accordion portions 28 of wall 20A, this outward force drives wall 20A to actually move outward whereby such accordion-like portions 28 change shape to a simple curve like the rest of wall 20A (as shown in FIG. 1D). Guides 99 serve to guide the outward movement of walls 21A and include a lock to hold such walls 21A in place in the expanded position.

Conversely, a movement of gears 94 in a reverse direction-which can be initiated by micro engines 91 as controlled by their ON/OFF switch-reverses the expansion of walls 21A and collapses walls 20A such that the walls 20A move inwardly. It is readily apparent that an outward movement of walls 20A increases a diameter and volume of the space within the walls 20A and that an inward movement decreases such diameter and volume. This is one non-limiting way in which adjustment mechanism 90 can operate.

In some implementations, the scanner bore is divided into at least two or at least three or all four modules from among (i) the head module 21, (ii) the thoracic module 22, (iii) the abdominal module 23 and (iv) the legs module 24.

One further embodiment of assembly 10 is a portable assembly for an MRI imaging system, the assembly comprising:
a scanner bore 20 having an inner wall;
electromagnets 40 positioned along the inner wall, each electromagnet comprising a metallic core surrounded by a coil assembly 44, the coil assembly 44 comprising one or more coils 44A, 44B etc. made of a metal and graphene;
a power supply 50 configured to generate pulses of electric current, at a specified repetition rate and for a specified duration, and an electromagnetic field;
detectors 70 positioned along the inner wall and configured to detect a signal derived from changes in a tissue of a subject inside the scanner bore, the changes responsive to the electromagnetic field, and to transmit the signal for use by a remote computer system in generating at least one of an image, an analysis and a diagnosis;
wherein the generated pulses are of a duration of a second or less.

Any of the other features may be incorporated into this embodiment. As one example, any version of cooling system 80 may be incorporated into this embodiment (or into any other embodiment that does not explicitly mention the cooling system 80). As another example, any version of the adjustment mechanism 90 may be incorporated into this embodiment.

As shown in FIG. 8A, each coil of the coil assembly 44 may comprise a metal layer, a graphene layer and a ceramic layer.

In some versions, each electromagnet 40 is superconductive. For example, the coil assembly 44 comprises at least two coils and liquid nitrogen is situated in a space between adjacent coils of the at least two coils 44A, 44B, etc. of coil assembly 44. This liquid nitrogen may be hermetically sealed in place. The liquid nitrogen may be at a temperature of minus 185 °C or higher, for example minus 183 °C. This provides an advantage over the prior art superconductive elements (for example those that utilize helium) where a temperature of minus 270 °C has to be reached in order to achieve superconductivity of the electromagnet. As a result of the superconductive effect, the resistance of the ceramic declines to zero. Since that happens to the ceramic it happens to the other materials of the tube of the coil of the electromagnet 40. An additional advantage of using liquid nitrogen to generate superconductive electromagnets for MRI imaging systems is that liquid nitrogen is cheap (in general) and abundant and in particular is far cheaper than helium.

At the time of the brief pulses (for example 32 milliseconds) the electromagnet 40 is akin to a permanent magnet, but this is achieved without the weight of a permanent magnet (i.e. without a permanent magnet).

In some embodiments, power supply 50 comprises piezoelectric crystals or other materials or pyro-piezoelectric materials or crystals, and an actuator. The power supply may be controlled by a controller so as to generate the pulses of electric current at the specified repetition rate and for the specified duration.

The duration of the pulses may be 50 milliseconds or less, 100 milliseconds or less, 250 milliseconds or less or 500 milliseconds or less, or may be from 20 milliseconds to 50 milliseconds, for example 32 milliseconds plus or minus 10%.

The number of pulses per second is 3 or is less than 3, or in other embodiments is less than 2.

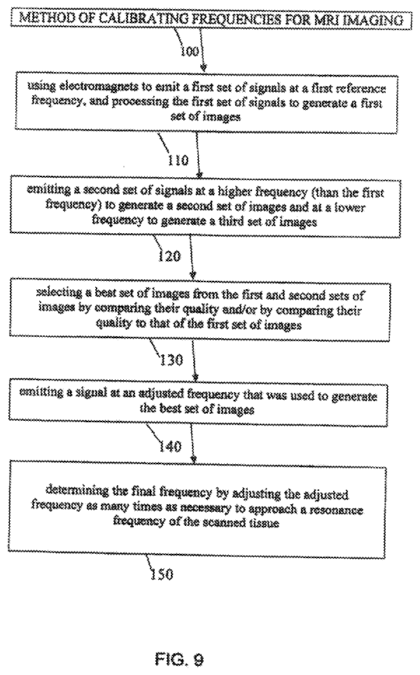

As shown in FIG. 9, another embodiment is a method 100 of calibrating frequencies for an MRI imaging system.

Method 100 may have a step 110 of using electromagnets 40 of a coil assembly 44 (or more than one coil assembly), for example a coil assembly 44 connected to an inner wall 26 of a scanner bore 20, to emit toward a subject a set of first signals, for example a first signal at a first frequency (for example a first radio wave frequency), at least two times, for example three times, the first frequency of the coils of the one or more coil assemblies 44, 45, 46, etc. selected based on previous MRI imaging experience or based on being accepted as a standard frequency for MRI imaging of the tissue of a particular body portion (or a subportion of that particular body portion) and processing the first (for example radio wave frequency) signal to generate at least two first images.

Let us suppose that we select 1.8 MHz as a starting reasonable frequency for one or more electromagnetic coils 44A, 44B (or 45A, 45B or 46A, 46B) of the coil assemblies 44, 45, 46, etc., for example based on our experience or because it is often used for a particular portion of the body being scanned, or for some other reason such as being one of the frequencies that is or is close to an average or median resonance frequency for such tissue. Let us further suppose that the tissue inside the subject's head has a resonance frequency of 1.5 MHz.

The resonance frequency is that frequency with the highest amplitude of the signal that reflects back from the subject's tissue and is detected by a sensor or detector. The closer one approaches the actual resonance frequency, the better the quality of the image produced. This is because as the amplitude of the signal increases, the signal is stronger and of better quality (everything else being equal).

Continuing with that example, in order to find the resonance frequency of the subject's tissue being scanned, for example inside the cranium (as one non-limiting example), a set of three images are taken at a frequency of the one or more coil assemblies 44, 45, 46, etc. that is considered a standard frequency for head MRI imaging—in this case 1.8 Mhz. “A standard frequency” is any frequency published as a standard frequency for MRI imaging of that particular body portion (or subportion) that is accepted by the medical profession in the locale where the MRI imaging is occurring, or based on any other reasonable criteria. In some embodiments, instead of a standard frequency that has been accepted, any reference frequency is used based on the supposition of the technician and/or a supervising physician. In this case, the “standard frequency” may be replaced by the “reference frequency” in the discussion below and in method 100 in general. In some embodiments, the standard frequency or the reference frequency is selected by an artificial intelligence software program in the remote computer or in a computer system attached to the assembly 10.

Then a set of three more images are taken at a coil assembly frequency of 2.0 MHz, a higher frequency than the standard frequency used and in addition a set of three more images are taken at 1.6 MHz, a lower frequency than the standard frequency used. One then compares the quality of the images taken at 1.6 MHz to those taken at 1.8 MHz, and compares the quality of the images taken at 2.0 MHz to those taken at 1.8 MHz. If the images taken at 2.0 MHz are worse than those taken at 1.8 MHz and the images taken at 1.6 MHz are better than those taken at 1.8 MHz, a simple pattern is detected, for example by the remote or local computer system. One would therefore conclude that it is wise to continue the experiment by sampling three more images taken at a frequency lower than 1.6 MHz, namely for example 1.5 MHz. This process is continued by trial and error with or without the assistance of machine learning.

Alternatively, since the range of frequencies of the coil assemblies is assumed to be from 0.1 MHz to 10 MHZ, one option is to simply image the subject at 0.1 MHz and then at a higher frequency such as 0.2 MHZ and then at a higher frequency of 0.3 MHZ until we find that the image quality that is the best (or we try 2 MHz, then 3 MHz, then 4MHz, then 5 MHz, then 6 MHz, then 7 MHz, then 8 MHz, then 9 MHz, then 10 MHz until we find the ideal image quality).

Raising the frequency of the one or more coil assemblies 44, 45, 46, etc. may improve image quality but if one raises the frequency too much (and lowers the wavelength too much) then the depth of penetration of the signal declines. For example, at a frequency of 1.0 MHz or 3.0 MHz, one may obtain clear images of deep areas of the brain as well as of the brain cortex and other superficial portions of the brain; whereas at a frequency of 5 MHz or 6 MHz one may obtain very clear images of the brain cortex and other superficial areas of the brain but less clear images of the deep areas of the brain.

To find the optimal frequency, use trial and error by either artificial intelligence or a human technician or physician.

The optimal frequency for one person is not the optimal frequency for another person because each person has a different distribution of water in their tissue.

Accordingly, method 100 may also comprise another step 120 of using the electromagnets 40 to emit toward the subject a second set of signals (for example radio wave frequency signals) at a frequency higher than the first frequency and a third set of signals at a frequency lower than the first frequency, and processing the second set of signals and the third set of signals to generate a second set of images and a third set of images.

A further step 130 of method 100 may be selecting a best set of images from among the second set of images and the third set of images by comparing a quality of the second set of images with a quality of the third set of images, or by comparing the first set of images with each of the second set and the third set of images. For example step 130 may be implemented by selecting a best set of images from among the second set of images and the third set of images by comparing a quality of the first, second and third sets of images, for example by comparing a quality of the first set of images to a quality of the second set of images and by comparing a quality of the third set of images to a quality of the first set of images. Alternatively, the second and third sets of images may be compared.

Method 100 may also have a step 140 of emitting a set of one or more signals at the adjusted frequency that was used to generate the best set of images (in step 130).

Step 150 of method 100 comprises determining a final frequency by adjusting the adjusted frequency as many times as necessary to approach a resonance frequency of the subject's tissue being scanned. This may be implemented by determining a final frequency by adjusting the adjusted frequency as many times as necessary by comparing as many times as necessary a quality of a set of images generated from a signal emitted at a most current adjusted frequency with a quality of a set of images generated from a signal emitted at a new frequency selected to be higher or lower than the most current adjusted frequency.

Step 150 may alternatively be implemented for example by comparing as many times as necessary a quality of an images generated from a signal emitted at a most current adjusted frequency with a quality of an image generated from a signal emitted at a new frequency higher than and at a new frequency lower than the current adjusted frequency to arrive at a best newly adjusted frequency. In some embodiments, the best new frequency can be designed to be consistent with a direction of the change in frequencies during the last (most recently adjusted) adjustment of the frequencies.

For example, step 150 may involve repeating the above steps 110, 120, 130, 140 as part of a trial and error process to arrive at a final specified frequency. For example, a fourth set of images and a third set of images are obtained and compared to the most recent “best” frequency“. The frequency of the image of better quality is then the new ”best“ frequency”. This continues until a final frequency is reached. One way of finalizing the frequency is determining when the image quality reaches a preset threshold, for example a threshold of image resolution or other quantitative measure of quality. Another way is for the technician or physician to conclude that the image quality is subjectively satisfactory. Still another way is that the adjustments in the frequency are becoming smaller and smaller (for example based on a preset percentage of the change in frequency).

In some embodiments, machine learning and artificial intelligence are used in one or more of steps 110, 120, 130 and 150. If machine learning is used, then, in some cases, a supervised training dataset is created by machine learning. In one non-limiting example, a binary classification is made by an algorithm in comparing the quality of images generating by new frequencies with the quality of images generated by the most current adjusted frequency.

Method 100 may also involve using a power supply connected to each of the electromagnets to generate pulses of electric current and an electromagnetic field, the power supply controlled by a controller so as to generate the pulses of electric current at a specified repetition rate and for a specified duration. The generated pulses may be of a duration of a second or less.

Method 100 may have a step of using sensors/detectors positioned along the inner wall 26 that are configured to detect a signal derived from changes in a tissue of a subject inside the scanner bore, the changes responsive to the electromagnetic field, and to transmit the signal for use by a remote computer system in generating at least one of an image, an analysis and a diagnosis.

As a result of the design of the assembly 10, including but not limited to the fact that a permanent magnet is not used, and therefore the cooling system is much simpler, and the fact that the computer system used for analysis and image generation may be remote (although in other embodiments it is not remote and is attached to or is part of assembly 10), assembly 10 may have a weight of 100 Kgs or less and may be portable. In some embodiments, the weight is 40 Kgs or less. In some embodiments in which graphene is used to replace metal coils, as described, the estimated weight of the entire assembly 10 is about 30 Kgs and the estimated weight of each module is 7.5 Kgs (plus or minus 10%).

In some embodiments, each coil has its own computer with an artificial intelligence system and transmits a signal to a common data system for the subsequent creation of a common data file and its transmission to common networks for imaging, examination, and diagnosis. The remote computer to which the data file is sent may also have software algorithms stored on a memory storage and configured to be executed by the remote computer system. These algorithms and/or software may include machine learning.

Outside the assembly 10, but part of the MRI scanning system, may be a hardware and software module 98 configured to interact with the detectors 70 (either directly as in FIG. 1D or indirectly through data acquisition and transmission unit 88 as in FIG. 1A and FIG. 1B) so as to provide remote control of a scanning by the scanning bore 20, including remote configuration of scanning parameters and programs.

In some embodiments, there is also a hardware and software module for the preparation of specially prepared water (electrochemically cleaned water to obtain pure water and optionally with nanoparticles of chromium, nickel, iron and/or neodymium) for cooling the coils and, subsequently, specially contrasting liquids (i.e. liquid nitrogen), including those with a reduced (or zero content) gadolinium content, based on this specially prepared water with dispensers and individual dosage calculation, as well as specially prepared water and liquids to facilitate, improve, and activate the patient's excretory systems after the use of specially contrasting liquids.

In some embodiments the MRI system (or assembly 10) includes a hardware and software module with detectors for creating portraits of the subject, such as temperature, humidity and/or electrical portraits of the nervous system activity of the subject, sound portraits, portraits of micro-motor activity, chemical portraits, pulse and respiration of the subject so as to be integrated with data from the MRI scan data files.

In one option, the electromagnets have an air cooling system including a Ranque-Hilsch vortex tube with an adapter that allows operation both from a compressed air cylinder and from various compressors. Other options have no compressor to diminish weight.

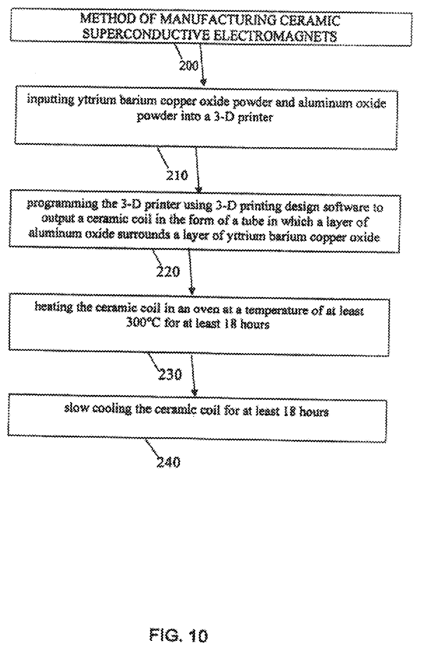

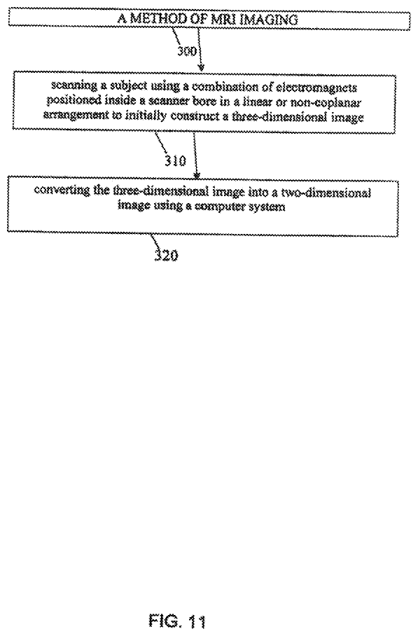

As shown in FIG. 10, a further embodiment is a method 200 of producing a ceramic superconductive coil for an electromagnet 40 for an assembly 10 of an MRI imaging system or machine, which in some implementations is portable. Method 200 may comprise a first step 210 of inputting yttrium barium copper oxide powder and aluminum oxide powder into a 3-D printer.

Step 220 may comprise programming the 3-D printer using 3-D printing design software to output a ceramic coil in the form of a tube in which a layer of aluminum oxide surrounds a layer of yttrium barium copper oxide.

Step 230 may comprise heating the ceramic coil in an oven at a temperature of at least 300 °C for at least 18 hours.

Step 240 may involve slow cooling the ceramic coil for at least 18 hours.

As shown in FIG. 1H1 (and FIG. 1A), the electromagnets 40 of assembly 10, in any embodiment, may be arranged such that the combination of three or more of these electromagnets 40 comprises multiple pairs or groups of electromagnets 40 (including coils) so as to generate three-dimensional images (initially). This was described with respect to assembly 10. In one non-limiting implementation shown in FIG. 1H1, a pair 40-40 of electromagnets 40 (called “fronto-parietal occipital” in FIG. 1H1) includes an electromagnet 40 positioned facing the frontal lobe and the second electromagnet of the pair positioned facing either the occipital lobe or the parietal lobe of the subject's brain. In a second pair 40-40 of electromagnets 40, one electromagnet 40 of a pair faces a left side of the temporal lobe and the other faces the right side of the temporal lobe. In a third pair 40-40 of electromagnets 40, one electromagnet 40 in the pair faces a left side of the frontal lobe and the other faces the right side of the frontal lobe. An array 40A of one or a group of electromagnets faces the parietal lobe. In a fourth pair 40-40 of electromagnets 40 (called “oblique fronto-parietal” in FIG. 1H1, one faces the left side of the frontal lobe and one electromagnet faces the right side of the parietal lobe. Due to the nonlinear positioning (for example circular positioning) of the electromagnets 40, the patient 15 or subject need not be moved by the assembly 10 in order to capture three-dimensional or multi-dimensional holographic images.

In the non-limiting implementation shown in FIG. 1H2, the electromagnets 40 containing the coils are arranged in multiple, for example three, substantially linear arrays 40A. The support structure 16 on which the patient 15 is resting is configured to be moved by an actuator through up to three degrees of freedom relative to the electromagnets 40, as shown by the three axes (x, y, z) in FIG. 1H2. As can be seen, two of the three arrays 40A form a pair 40A-40A.

As a result of either the implementation shown in FIG. 1H1 or that shown in FIG. 1H2, the electromagnets 40 with their coils are able to emit a signal that generates a three dimensional image rather than a two dimensional image. Accordingly, in some embodiments, the image generated by the remote computer system is initially a 3-dimensional image, in contrast to the prior art, which does not directly generate a three-dimensional or multi-dimensional holographic image directly (but rather starts with a two-dimensional image that it has to first convert to a three-dimensional image). In that case, the remote computer system 98 then converts the holographic image into a two dimensional image. In this conversion process from 3-D to 2-D, the partial voluming artifact common to MRI imaging is significantly reduced and localization of a defect in the subject's body is more precise. Precise localization of a defect is particularly critical for brain injury. This localization is improved using the MRI imaging system or assembly 10 of the embodiments described herein in embodiments in which the initially obtained image is a three-dimensional image sent to remote computer system 98.

This contrasts with what current MRI imaging systems do, which is to begin with a 2-D image that is converted to 3-D. These systems do this because they use just two emitters to form the initial image or else they use more than two emitters and these three or more emitters are positioned in a linear configuration.

Accordingly, as shown in FIG. 11, one embodiment is a method 300 of imaging using an MRI imaging system, which may be used in any of the heretofore described embodiments. Method step 310 comprises scanning a portion of a subject using nonlinear or noncoplanar electromagnets 40 positioned (for example along an inner wall 26 of a scanner bore 20) such that a combination of the electromagnets 40 comprises multiple pairs or groups of electromagnets 40 positioned, for example as shown in FIG. 1H1 or FIG. 1H2, so as to generate three-dimensional images. The combination of such electromagnets 40 arranged in this way is configured to emit a signal to tissue of the subject, wherein a signal derived from nuclear magnetic resonance of tissue of the subject is detected (for example by detectors 70) and is transmitted to a computer system 98 (which may be remote) to initially construct a three-dimensional image.

Step 320 comprises converting, by the computer system, the three-dimensional image into a two-dimensional image.

A further step may comprise outputting, by the computer system, one or more of (i) an analysis of the two-dimensional image and (ii) a diagnosis based on the two-dimensional image.

In some versions, the quantity of the electromagnets 40 may range from 16 to 256.

In some versions, each of the electromagnets 40 may comprise a coil assembly 44.

Some versions of method 300 may involve using a power supply 50 connected to each of the electromagnets 40 to generate pulses of electric current and an electromagnetic field, the power supply controlled by a controller so as to generate the pulses of electric current at a specified repetition rate and for a specified duration, wherein the generated pulses are of a duration of a second or less.

In some versions, the computer system may be a remote computer system 98 and the electromagnets 40 may be part of a portable assembly 10 in accordance with any of the embodiments described herein or may be part of an assembly 10 for a stationary MRI machine in other embodiments.

Any of the details of the embodiments described herein may be combined with method 300.

In some versions, method 300 may be used also with a stationary MRI imaging system.

One embodiment is a method of imaging using an MRI imaging system, which may be used in any of the heretofore described embodiments. This method may include a step comprises scanning a portion of a subject using electromagnets 40 positioned along an inner wall 26 of a scanner bore 20 such that a combination of the electromagnets 40 is not colinear, and the combination emits a signal to tissue of the subject, wherein a signal derived from nuclear magnetic resonance of tissue of the subject is detected (for example by detectors 70) and is transmitted to a computer system 98 (which may be remote) to initially construct a three-dimensional image.

A second step in this method comprises converting, by the computer system, the three-dimensional image into a two-dimensional image.

A further step may comprise outputting, by the computer system, one or more of (i) an analysis of the two-dimensional image and (ii) a diagnosis based on the two-dimensional image.

This method is identical to method 300 in all other respects (i.e. in all respects other than the configuration of the electromagnets being not colinear instead of being not coplanar).

In certain embodiments, a video is output by the MRI assembly 10. From the MRI video output, particular collections of still frames can be extracted, such as an MRI protocol for Alzheimer's, an MRI protocol for Parkinson's or any other MRI protocol for any other medical condition or suspected medical condition. This can refer to a full body MRI protocol or an MRI protocol of a particular organ of the body or of a region of the body (for example abdominal region).

For example, if the patient originally was diagnosed with or suspected of having brain atrophy and a video MRI protocol was run, this would also include all other brain MRI protocols including for example the MRI protocol of Alzheimer's. Therefore, if several years later the patient complains of memory loss, the physician need not order a new MRI. Instead, the physician can simply request an extraction of the MRI protocol for Alzheimer's (for example) from the already-existing MRI video data.

The MRI video also allows for easier comparison of the patient's condition against a reference value taken in the past. Suppose the patient had an MRI due to a brain injury and five years later has an MRI due to memory loss. In the first case, the MRI protocol would have been for traumatic brain injury (TBI) and in the second case it would have been for Alzheimer's. But currently no effective comparison is available to be made between the patient's current memory conditions and his past memory condition from five years ago. In contrast, if he had a brain injury he would have received a video MRI which in certain embodiments covers all brain MRI protocols so that if he now develops memory loss he will have a video MRI that in certain embodiments also includes all brain MRI protocols. Thus, a comparison can be made between his current and past conditions simply by now requesting that the MRI protocol for memory loss (Alzheimer's for example) be extracted from the old video from five years ago. This can be a very helpful diagnostic tool for the physician.

In some embodiments, each of the electromagnets 40 is formed as a coil assembly 44 and each of the coil assemblies 44, 45, 46 etc. comprise two or three or four or more inter-nested coils (for example coils 44A, 44AA, 44AAA, 44B), each configured to carry pulses of electric current at a different frequency. This may include an outer coil 44B and a first inner coil 44A (and it make include additional inner coils 44AA and/or 44AAA and/or others), such that the outer coil 44B of one particular coil assembly at least partially shields the electromagnetic fields generated by the first inner coil of the one particular coil assembly from background electromagnetic fields of coils of other adjacent coil assemblies.

The controller 60 and power supply 50 may be connected to each coil of the coil assembly 44 independently. This results in the fact that each coil may have its own voltage, amperage, magnetic field and gradient. This arrangement, in some embodiments, magnifies the signal to noise ratio in some cases by eight to ten times.

In certain embodiments, the video MRI session yields real time pulsations and graphs and tissue of brain, arteries, veins of brain, ventricles, glymphatic system and arterial to venous shunting transfer time without contrast. Currently, arterial to venous shunting transfer time is performed with contrast and achieves a temporal resolution of 50-100 milliseconds, which is called functional MRI. The glymphatic system is not discernable from current MRIs. Certain embodiment of the video MRI will achieve a resolution of up to (meaning the resolution quality is “as good as”) one to five milliseconds.

Today, MRI protocols do not include oblique sectional views of the brain and other regions of the subject. Although it is possible, it would take an inordinate amount of time. In some embodiments herein of the video MRI assembly, it would be possible to generate oblique views and include these views in the video MRI protocol (which essentially means all MRI protocols.

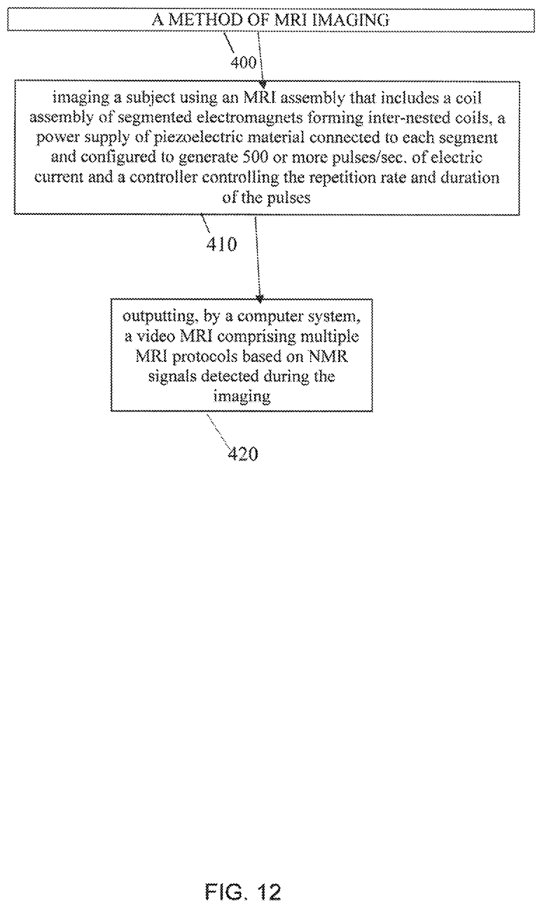

As shown in FIG. 12, method 400 is a method of generating a video MRI that includes multiple MRI protocols. Method 400 may include a step 410 of imaging a subject using an MRI assembly 10 that includes a coil assembly of segmented electromagnets forming inter-nested coils, a power supply, including a piezoelectric material and an actuator, independently connected to each segment and configured to generate pulses of electric current at a repetition rate of five-hundred or more pulses per second and a controller controlling the repetition rate and duration of the pulses.

Method 400 may also comprise a step 420 of outputting, by a computer system, a video MRI comprising multiple MRI protocols based on NMR signals detected during the imaging

For example, step 410 may comprise imaging a subject using an MRI assembly that includes electromagnets, each formed as a coil assembly, each coil assembly of which may include from two to twelve segments, wherein the assembly also includes:
a power supply connected to the electromagnets and configured to generate pulses of electric current at a repetition rate of five-hundred or more pulses per second, the power supply comprising piezoelectric material and an actuator, and controlled by a controller so as to generate the pulses of electric current at the repetition rate and for a duration specified by the controller;
detectors configured to detect a nuclear magnetic resonance (NMR) signal from tissue of the subject, and to transmit the NMR signal for use by a computer system to generate a video image of the subject,
wherein each of the coil assemblies comprise at least two inter-nested coils, each of the at least two inter-nested coils of each of the coil assemblies includes an outer coil and at least a first inner coil and is configured to carry pulses of electric current at a same or a different electromagnetic frequency, such that the outer coil of one particular coil assembly at least partially shields the electromagnetic fields generated by at least the first inner coil of the one particular coil assembly from background electromagnetic fields of coils of other adjacent coil assemblies.

Method 400 may further comprise a step of extracting from the video MRI a particular MRI protocol using known software like Osirix, 3D Slicer and Horos. For example, the video MRI may comprise at least seven different MRI protocols that are neurological or brain protocols and for example if a new symptom develops (for example after a significant apse in time since the MRI) any of these protocols may be extracted later, as needed. In another example, the video MRI encompasses at least four different MRI protocols that are musculoskeletal protocols and any of these protocols may be extracted if for example a new symptom develops. In another example, the video MRI encompasses at least four different MRI protocols that are abdomen and pelvis protocols and any of these protocols may later be extracted as needed. In a still further example, the video MRI encompasses at least five different MRI protocols that are head, neck or vascular protocols, any of which may later be extracted for example if for example a new symptom develops or for other reasons. In a yet further example, the video MRI encompasses at least five different MRI protocols that are cardiac protocols that may be extracted later from the MRI video image for example if a new symptom develops. In another example, the video MRI encompasses at least two different MRI protocols that are breast protocols, which may be extracted.

In some embodiments, a duration of the imaging session that produces the video MRI is 90 seconds or less (for example 60-90 seconds) for sessions in which the imaging is of the whole body of the subject. In certain embodiments, a duration of the imaging session that generates the video MRI is 30 seconds or less (for example 15-30 seconds) for sessions in which the imaging is of an organ or a region of the body of the subject.

In explaining the shielding by the outer coils of the inner coils, Applicant defines “coil” to mean a separate winding or a separate conducting/superconducting circuit designed to generate, modify, compensate for, or record a magnetic and/or electromagnetic field, and defines “coil assembly,” to mean a multilayer module consisting of several nested coil layers. In one implementation, the coil assembly contains three, four or five or more such layers, arranged in a nested structure. Geometrically, such a coil assembly 44, 45, 46 etc. may have, for example, the shape of an elongated, triangular, truncated pyramidal structure, or other shapes such as a frustoconical shape. Each coil layer, in turn, can be divided into segments, for example, two to twelve or three to twelve segments, oriented along and/or perpendicular to the longitudinal axis of the coil assembly. A central rod or core of similar proportions, such as an elongated triangular pyramid with a truncated apex, may be located within the coil array. This rod may be made of a special alloy or composite material designed to structure, direct, concentrate, redistribute, and/or “focus” the magnetic field generated by the coil array. In other words, it may act as an internal magneto-forming, magnetically guiding, magnetically conducting, stabilizing, and/or matching element, working in conjunction with the coil layers and their segments. Applicant further defines “coil array” to refer to a collection of several such coil assemblies arranged around the object under study (e.g. the subject) and working in concert. A system may use, for example, from two to sixty-four coil assemblies 44, 45, 46, 47 etc.

Without being bound by theory, Applicant in general understands that outer coil 44B (or 45B, 46B, etc.) acts as an at least partially shielding and compensating element for the inner coils 44A, 44AA etc. (or 45A, 45AA, etc., 46A, 46AA, etc.) of other coil arrays and coil assemblies 44, 45, 46, etc. as a result of position and location of the coils and electrical insulation of the outer and inner coils.

For example, since the outer coil layers are located outside the inner coil layers, they are located between the inner coils of a given coil array and adjacent coil arrays (and between the inner coils of a given coil assembly and adjacent coil assemblies). Therefore, they can act as an intermediate protective, compensating, and matching layer.

In addition, the outer and inner coils are electrically isolated from each other. This isolation is necessary to prevent breakdown, parasitic currents, and unwanted electrical coupling between layers, as well as to allow independent control of different layers and segments. This allows the outer layers to operate independently from the inner layers and be used not only as working coils but also as compensating or shielding elements.

Furthermore, the outer coils or outer coil layers can be supplemented with magnetic shielding layers, such as Mu-metal foil, permalloy, or another material with high magnetic permeability. Such materials can partially deflect, redistribute, or attenuate external magnetic influences and stray fields emanating from adjacent coil assemblies. Such magnetic shielding layers can be separated from the windings by dielectric, thermally conductive, and/or cryogenically resistant insulating layers.

An additional factor is that the outer coil layer can function not only as a passive shield but also as an active compensation layer. That is, it can be supplied with a current of a specific shape, phase, and direction that partially compensates for the unwanted fields of adjacent coil assemblies, reduces cross-inductive coupling, and mitigates the impact of external electromagnetic interference on the internal coils of a given coil assembly.

Also, the central rod or core can also help regulate the field within the coil assembly. It can help direct and stabilize the magnetic flux, reduce field leakage, improve the matching of the inner and outer coil layers, and enhance the controllability of the resulting magnetic configuration. This allows the outer layers, inner layers, and central rod to function as a single magneto-forming system rather than as separate elements.

Optionally, the central rod or its associated channel can be adapted to supply, direct, or spatially align an external flux of muons or other probe particles from an external source, such as a temporarily connected or temporarily rented muon generator. In this embodiment, the rod can serve not only as a magneto-forming element but also as a guiding, collimating, matching, or interface element for integrating magnetic resonance imaging with an additional probe particle flux.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. An assembly for an MRI video imaging system, the assembly comprising:
a subject-receiving region having at least one surface;
electromagnets formed as a coil assembly and configured to generate an electromagnetic field, the coil assembly configured to emit a radio frequency (RF) signal to the subject;
a power supply connected to the electromagnets and configured to generate pulses of electric current, the power supply comprising piezoelectric material and an actuator, and controlled by a controller so as to generate the pulses of electric current at a repetition rate and for a duration specified by the controller;
detectors configured to detect a nuclear magnetic resonance (NMR) signal from tissue of the subject, and to transmit the NMR signal for use by a computer system in generating a video image that implements multiple MRI protocols,
wherein each coil assembly comprises at least two inter-nested coils, each of the at least two inter-nested coils configured to carry pulses of electric current at the same or a different electromagnetic frequency and including an outer coil and at least a first inner coil, such that the outer coil of one particular coil assembly at least partially shields the electromagnetic fields generated by at least the first inner coil of the one particular coil assembly from background electromagnetic fields of coils of other adjacent coil assemblies,
each coil assembly has at least two segments, each of the segments connected independently to the power supply.

2. The assembly of claim 1, wherein each of the coil assemblies comprise three or four inter-nested coils.

3. The assembly of claim 1, wherein the video image includes at least one full MRI protocol of a whole body of the subject and a duration of an imaging session during which the NMR signals used to generate the video image are acquired does not exceed 90 seconds.

4. The assembly of claim 1, wherein the video image covers at least one full MRI protocol of an organ or a region of the subject and a duration of an imaging session during which the NMR signals used to generate the video image are acquired does not exceed 30 seconds.

5. The assembly of claim 1, wherein each coil assembly has from two to twelve segments.

6. The assembly of claim 1, wherein the power supply is configured to generate the pulses at a particular repetition rate from 1000 to 10000 pulses per second.

7. The assembly of claim 1, wherein the power supply is configured to produce a current of 10 to 399 amperes.

8. The assembly of claim 1, wherein the power supply is configured to produce a voltage of from 10 volts to 10 kilovolts.

9. The assembly of claim 1, wherein the power supply is configured to generate 1-3000 kilowatts.

10. The assembly of claim 1, wherein a frequency of pulses of electric current transmitted to each coil assembly of the electromagnets is from 100 KHz to 10 MHz.

11. The assembly of claim 1, wherein the duration of each of the generated pulses is ten milliseconds or less.

12. The assembly of claim 1, wherein the duration of each of the generated pulses is 3.3 milliseconds to 333 milliseconds.

13. The assembly of claim 1, wherein a repetition rate of the pulses of the electric current transmitted to each coil assembly of the electromagnets is independently controlled by the controller.

14. The assembly of claim 1, wherein each of the coils of the electromagnets is shaped substantially frustoconical or shaped substantially as a triangular pyramid whose vertex has been truncated.

15. The assembly of claim 1, wherein the assembly is a portable assembly and a total weight of the portable assembly is less than 100 kilograms.

16. The assembly of claim 1, wherein the assembly is a portable assembly and a total weight of the portable assembly is less than 30 kilograms.

17. The assembly of claim 1, wherein the assembly comprises one or more of the following modules and a total weight of one of the following modules is less than 10 kilograms: (i) a head module, (ii) an abdominal module, (iii) a thorax module and (iv) a legs module.

18. The assembly of claim 1, further comprising a cooling fluid flowing through the coil or coil assembly of the electromagnets and further comprising metal nanoparticles in the fluid configured to increase a signal to noise ratio for the transmitted signal.

19. The assembly of claim 1, wherein the power supply comprises a separate element of piezoelectric material for each of the electromagnets.

20. A method of generating a video MRI that includes multiple MRI protocols, comprising:
imaging a subject using an MRI assembly that includes a coil assembly of segmented electromagnets of inter-nested coils, a power supply, including a piezoelectric material and an actuator, independently connected to each segment of the segmented electromagnets and configured to generate pulses of electric current at a repetition rate of five-hundred or more pulses per second, a controller controlling the repetition rate and duration of the pulses; and
outputting, by a computer system, a video MRI comprising multiple MRI protocols based on NMR signals detected during the imaging.

21. The method of claim 20, wherein each of the coil assemblies comprise at least two inter-nested coils, each of the at least two inter-nested coils of each of the coil assemblies includes an outer coil and at least a first inner coil and is configured to carry pulses of electric current at a same or a different electromagnetic frequency, such that the outer coil of one particular coil assembly at least partially shields the electromagnetic fields generated by at least the first inner coil of the one particular coil assembly from background electromagnetic fields of coils of other adjacent coil assemblies.

22. The method of claim 20, further comprising extracting from the video MRI a particular MRI protocol.

23. The method claim 20, wherein the video MRI comprises at least seven different MRI protocols that are neurological or brain protocols.

24. The method of claim 20, wherein the video MRI encompasses at least four different MRI protocols that are musculoskeletal protocols.

25. The method of claim 20, wherein the video MRI encompasses at least four different MRI protocols that are abdomen and pelvis protocols.

26. The method of claim 20, wherein the video MRI encompasses at least five different MRI protocols that are head, neck or vascular protocols.

27. The method of claim 20, wherein the video MRI encompasses at least five different MRI protocols that are cardiac protocols.

28. The method of claim 20, wherein the video MRI encompasses at least two different MRI protocols that are breast protocols.

29. The method of claim 20, wherein a duration of an imaging session during which the NMR signals used to generate the video image are acquired does not exceed 90 seconds and the video MRI is of a whole body of the subject.

30. The method of claim 20, wherein a duration of an imaging session during which the NMR signals used to generate the video image are acquired does not exceed 30 seconds and the video MRI is of an organ or a region of the subject.
